SEQUENCE LISTING (16 sequences)

SEQ ID NO: 1 - DNA, 14 bp, Artificial Sequence
  PNA SEQUENCE OF SENSING MODULE
natcaacactgcan

SEQ ID NO: 2 - DNA, 38 bp, Artificial Sequence
  DNA SEQUENCE OF THE NORMALIZING MODULE
tatatataggatcttgctgtctggtgtgcagtgttgat

SEQ ID NO: 3 - DNA, 26 bp, Artificial Sequence
  DNA SEQUENCE OF THE TARGETING MODULE
caccagacagcaagatcctatatata

SEQ ID NO: 4 - DNA, 69 bp, Artificial Sequence
  TARGETING MODULE WITH RNA APTAMER
caccagacagcaagatcctatatatagggggaucaauccaagggacccggaaacgcucccuuacacccc

SEQ ID NO: 5 - DNA, 58 bp, Artificial Sequence
  NUCLEIC ACID SCAFFOLD DESIGN FOR OPTIMIZATION OF SENSOR
ccctaaccctaaccctaacccgactcactgtttgtctgtcgttctaggatatatattt

SEQ ID NO: 6 - DNA, 57 bp, Artificial Sequence
  NUCLEIC ACID SCAFFOLD DESIGN FOR OPTIMIZATION OF SENSOR
atatatatcctagaacgacagacaaacagtgagtctttgttatgtgttatgtgttat

SEQ ID NO: 7 - DNA, 59 bp, Artificial Sequence
  NUCLEIC ACID SCAFFOLD DESIGN FOR OPTIMIZATION OF SENSOR
aaatatatatcctagaacgacagacaaacagtgagtctttgttatgtgttatgtgttat

SEQ ID NO: 8 - DNA, 56 bp, Artificial Sequence
  NUCLEIC ACID SCAFFOLD DESIGN FOR OPTIMIZATION OF SENSOR
ccctaaccctaaccctaacccgactcactgtttgtctgtcgttctcggatatatat

SEQ ID NO: 9 - DNA, 57 bp, Artificial Sequence
  NUCLEIC ACID SCAFFOLD DESIGN FOR OPTIMIZATION OF SENSOR
atatatatccgagaacgacagacaaacagtgagtctttgttatgtgttatgtgttat

SEQ ID NO: 10 - DNA, 56 bp, Artificial Sequence
  NUCLEIC ACID SCAFFOLD DESIGN FOR OPTIMIZATION OF SENSOR
atatatatccggaacgacagacaaacagtgagtctttgttatgtgttatgtgttat

SEQ ID NO: 11 - DNA, 55 bp, Artificial Sequence
  NUCLEIC ACID SCAFFOLD DESIGN FOR OPTIMIZATION OF SENSOR
atatatatccgaacgacagacaaacagtgagtctttgttatgtgttatgtgttat

SEQ ID NO: 12 - DNA, 54 bp, Artificial Sequence
  NUCLEIC ACID SCAFFOLD DESIGN FOR OPTIMIZATION OF SENSOR
atatatatccaacgacagacaaacagtgagtctttgttatgtgttatgtgttat

SEQ ID NO: 13 - RNA, 43 nt, Artificial Sequence
  RNA APTAMER
gggggaucaauccaagggacccggaaacgcucccuuacacccc

SEQ ID NO: 14 - DNA, 32 bp, Artificial Sequence
  OLIGONUCLEOTIDE O1 FOR I SWITCH SENSOR
ccccaaccccaatacattttacgcctggtgcc

SEQ ID NO: 15 - DNA, 32 bp, Artificial Sequence
  OLIGONUCLEOTIDE O2 FOR I SWITCH SENSOR
ccgaccgcaggatcctataaaaccccaacccc

SEQ ID NO: 16 - DNA, 41 bp, Artificial Sequence
  OLIGONUCLEOTIDE O3 FOR I SWITCH SENSOR
ttataggatcctgcggtcggaggcaccaggcgtaaaatgta

(19) United States
(12) Patent Application Publication
(10) Pub. No.: US 2017/0101669 A1
(43) Pub. Date: Apr. 13, 2017

(54) Nucleic Acid Based Sensor and Methods Thereof

(71) Applicant: National Centre for Biological Sciences, Banglore, Karnataka (IN)

(72) Inventors: Yamuna KRISHNAN, Bangalore (IN); Sonali SAHA, District Cooch Behar (IN); Ved PRAKASH, Ranchi, Jharkhand (IN)

(21) Appl. No.: 15/129,833

(22) PCT Filed: Apr. 15, 2014
(86) PCT No.: PCT/IB2014/060736

§ 371 Date: Sep. 28, 2016

(51) Int. Cl. C12Q 1/68

(52) U.S. Cl. CPC C12Q 1/6825

(57) ABSTRACT

The present disclosure relates to a nucleic acid based sensor, comprising a sensing 5 module, a normalizing module and a targeting module. It also relates to a method of obtaining and targeting the sensor and its use to identify and optionally quantify a target in a specific cellular microenvironment.

### TECHNICAL FIELD

The present disclosure relates to a nucleic acid based, fluorescent ratio-metric sensor comprising a sensing module, a normalizing module and a targeting module. It also relates to a method of obtaining the sensor, targeting the sensor and its use to identify and optionally quantify a target in a specific cellular microenvironment.

### BACKGROUND AND PRIOR ART OF THE DISCLOSURE

Chloride is a major physiological anion. It is known that chloride ion accumulation in endosomal compartment provides principal electrical shunt to permit endosomal acidification. Members of ubiquitously expressed CLC protein family of chloride channels and transporters play important roles in regulating endosomal chloride ion concentration and pH. Among them, ClC-6 and ClC-7 chloride channels are known to reside in late endosome and lysosome. It has been shown that ClC-6 and ClC-7 knockout mice display neuro-degeneration and severe lysosomal storage diseases, despite unchanged lysosomal pH in cultured neurons. This indicates an important role of intracellular chloride ion that is beyond maintaining pH homeostasis.

However, due to lack of suitable probes to specifically localize chloride sensors in well-defined cellular micro-environments, the role of intracellular chloride in specific pathways is not well elucidated. Moreover, exact measurements of lysosomal Chloride ion [Cl⁻] concentration have not been reported due to the lack of suitable Chloride ion (Cl⁻) sensors for high Chloride ion (Cl⁻) concentration at low pH. Hence, it is important to develop a fluorescent ratio-metric sensor for quantitative chloride ion measurement at specific locations inside the cell.

Existing strategy in the prior art for designing DNA based sensors is based on DNA recognition motif for the specific chemical entity. Till date, no DNA motif has been reported to recognize chloride ion. Therefore, the existing strategy is not suitable for designing chloride ion sensor.

For most of the biologically important ions, small molecule sensing technology is already available. There are two disadvantages with small molecule sensing technology:
a) extrusion of the indicators from the cell; and
b) difficulty to achieve specific and uniform localization of the indicators within the different microenvironments of the cell.

Further, I) Small molecule indicators, such as Cl⁻ indicators are loaded into cells by long-term incubation (up to about eight hours) in the presence of a large excess of dye or by brief hypotonic permeabilization. As membranes are slightly permeable to the indicator, rapid leakage may occur. Experimentally determined estimates of leakage vary quite widely. II) Since these dyes are diffused across the cell, they provide global and low spatial resolution, diffused images of ionic environments inside the cell of interest.

Presently in the prior art, ratiometric imaging for chloride ion measurement is based on Dextran conjugated to BAC (10, 10′-Bis[3-carboxypropyl]-9,9′-biacridinium Dinitrate) and chloride insensitive TMR (Tetramethylrhodamine). The conjugate is called BAC-TMR-dextran. In this strategy, ratio of BAC and TMR fluorescence cannot be controlled due to variable degree of labeling of each fluorophore on dextran. Moreover, emission of TMR has high spectral overlap with emission of BAC. Therefore, image analysis may turn out to be complicated. Some of the disadvantages of this process are given below:

1. With BAC-Dextran, molar labeling ratio of biacridinium (BAC):TMR:dextran is not controllable from batch to batch. This is because, the degree of labeling on Dextran varies between two to six sites per dextran for 40 kDa dextran as seen in prior art.

2. Dextran cannot be targeted or restricted along a specific endocytic pathway without functionalization to an endocytic ligand. So, it is only used to probe those pathways where:

(i) the pathway has a ligand;

(ii) the ligand can be functionalized to dextran; and

(iii) functionalization does not change the trafficking properties of the ligand.

3. DNA nanostructures are used to probe the environment of any trafficking protein that traffics via the plasma membrane.

4. Further, targeting BAC-TMR-dextran to a compartment of choice requires BAC-dextran to be conjugated to a different protein every time.

Disadvantages of this technology are that a) it increases the overall size of the sensor and b) multiple reaction steps involved decrease yield of the sensor. However, according to targeting strategy of the present disclosure, no such modification is required.

5. Also, BAC-dextran is conjugated to protein using SPDP linker (N-Succinimidyl 3-[2-pyridyldithio]-propionate) which contains disulphide bond. Depending on the chemical environment inside the cell, this disulphide linkage may break and lead to mis-localization of the BAC-dextran.

The present disclosure overcomes the drawbacks faced in the prior art and provides for a nucleic-based sensor for target, including ions.

The existing genetically encodable sensors for chloride ion in the prior art are based on proteins which undergo static quenching by chloride, and their chloride sensitivity is pH dependent. Hence, physiological change in pH may affect the chloride ion concentration measurement. The present disclosure is based on small molecule sensing technology which is independent of physiological change in pH.

The nucleic acid based, fluorescent ratio-metric Chloride ion (Cl⁻) sensor, developed in a preferred embodiment of the present disclosure, undergoes receptor mediated endocytosis via Anionic Ligand Binding Receptor (ALBR) and Transferrin receptor. Upon internalization, the sensor specifically localizes in various endosomal compartments along the endo-lysosomal and recycling pathways. This sensor is also suitable for general targeting strategy to different sub-cellular compartments. The sensor remains associated with the receptors in these compartments. In addition to that, as membranes are not permeable for negatively charged DNA backbone, the small molecule Chloride ion (Cl⁻) indicator of the present disclosure does not leak out or diffuse across the cell.

### STATEMENT OF THE DISCLOSURE

Accordingly, the present disclosure relates to a nucleic acid based sensor comprising—a) sensing module comprising Peptide Nucleic Acid (PNA) strand and target sensitive molecule, b) normalizing module comprising nucleic acid sequence complementary to the PNA strand and target insensitive fluorophore, and c) targeting module comprising nucleic acid sequence complementary to the nucleic acid sequence of the normalizing module, optionally with aptamer; a method of obtaining nucleic acid based sensor as above, said method comprising acts of—a) obtaining sensing module by conjugating target sensitive molecule to Peptide Nucleic Acid (PNA) strand, b) obtaining normalizing module by conjugating target insensitive fluorophore to nucleic acid sequence complementary to the PNA strand of the sensing module, c) obtaining targeting module comprising nucleic acid sequence complementary to the nucleic acid sequence of the normalizing module, and optionally conjugating with aptamer, and d) combining the sensing, the normalizing and the targeting module to obtain the nucleic acid based sensor; a method of identifying and optionally quantifying target in a sample, said method comprising acts of—a) contacting and incubating the sample with nucleic acid based sensor as above, b) identifying the target by determining change in fluorescence level, and c) optionally quantifying the target by determining the fluorescence ratio of target insensitive fluorophore to target sensitive molecule; a method of targeting nucleic acid based sensor as above, said method comprising acts of—a) obtaining nucleic acid based sensor by method as above, b) adding the sensor to cell for cellular uptake, to obtain a cell with the sensor, and c) incubating the cell obtained in step b) for the nucleic acid based sensor to follow targeted cellular pathway within the cell; a kit for obtaining or targeting nucleic acid based sensor as above or identifying and optionally quantifying target in a sample, said kit comprising components selected from group of sensing module, targeting module, normalizing module, nucleic acid based sensor, cell, sample and instructions manual or any combinations thereof; and a method of assembling a kit as above, said method comprising act of combining components selected from group comprising sensing module, targeting module, normalizing module, nucleic acid based sensor, cell, sample and instructions manual or any combinations thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where:

FIG. 1 depicts the excitation and emission spectra of BAC, TMR and Alexa 647 fluorophores.

FIG. 2 depicts a plot showing chloride sensitivity of Clensor at different physiological pH.

FIG. 3 depicts the tabular and pictorial representation showing the effect of negatively charged backbone of DNA on chloride sensitivity.

FIG. 4 depicts the plot showing the linear range (about 0-200 mM) of chloride sensitivity of Clensor at pH 5.

FIG. 5 depicts in (a-c) Co-localization of Clensor with endosomal-lysosomal markers at indicated time points confirming the targeting of Clensor in this pathway. (d) Live cell chloride ion concentration measurement in early endosome, late endosomes and lysosomes. e) Co-localization of Clensor with recycling endosome marker Transferrin confirms the targeting of Clensor^{Tf} in this pathway. (f) Chloride ion concentration measurement in recycling endosomes of S2R+ cells.

FIG. 6 depicts localization of ClC family chloride transporters in Drosophila (DmClC) within endosomes along the ALBR pathway.

FIG. 7 depicts schematic representation of Clensor (a) and schematic representation of different molecularly non-identical species available in a BAC-TMR-dextran sample (b).

FIG. 8 depicts measurement of Chloride (Cl⁻) ion concentration in recycling endosome of S2R⁺ cells and in the background of RNAi knock down of DmClC-c and DmClC-b.

FIG. 9 depicts formation of Clensor and Clensor^{Tf} by gel mobility shift assay.

FIG. 10 depicts the representative image of hemocytes pulsed with Clensor (a), and graph representing competition experiment with excess unlabeled mBSA (b).

FIG. 11 depicts the design of Clensor^{Tf} (a), Representative image of S2R+ cells pulsed with Clensor^{Tf} (b), and graph representing competition experiment with excess unlabeled transferrin (c).

FIG. 12 depicts Clamping hemocytes (pulsed with Clensor) at known concentrations of chloride (a), Histogram showing R/G ratios of endosomes when clamped at different chloride concentrations (b), and in vitro and intracellular chloride calibration profile for Clensor (c).

FIG. 13 depicts Histogram showing the shift in R/G ratios of endosomes undergoing maturation.

FIG. 14 depicts measured chloride concentrations in the endosomal compartments with and without chloride channel blocker NPPB.

FIG. 15 depicts percentage co-localization of Clensor with different endosomal markers at given time points.

FIG. 16 depicts change in chloride concentrations and pH of the endosomal compartments during endosomal maturation with time (a), and representative images of hemocytes pulsed with Clensor before and after Bafilomycin treatment (b).

FIG. 17 depicts a graph representing the Chloride ion concentration in lifetime measurement mode.

### DETAILED DESCRIPTION OF THE DISCLOSURE

The present disclosure relates to a nucleic acid based sensor comprising:
a) sensing module comprising Peptide Nucleic Acid (PNA) strand and target sensitive molecule;
b) normalizing module comprising nucleic acid sequence complementary to the PNA strand and target insensitive fluorophore; and
c) targeting module comprising nucleic acid sequence complementary to the nucleic acid sequence of the normalizing module, optionally with aptamer.

The present disclosure also relates to a method of obtaining nucleic acid based sensor as above, said method comprising acts of:
a) obtaining sensing module by conjugating target sensitive molecule to Peptide Nucleic Acid (PNA) strand;
b) obtaining normalizing module by conjugating target insensitive fluorophore to nucleic acid sequence complementary to the PNA strand of the sensing module;
c) obtaining targeting module comprising nucleic acid sequence complementary to the nucleic acid sequence of the normalizing module, and optionally conjugating with aptamer; and
d) combining the sensing, the normalizing and the targeting module to obtain the nucleic acid based sensor.

The present disclosure also relates to a method of identifying and optionally quantifying target in a sample, said method comprising acts of:
a) contacting and incubating the sample with nucleic acid based sensor as above;
b) identifying the target by determining change in fluorescence level; and
c) optionally quantifying the target by determining the fluorescence ratio of target insensitive fluorophore to target sensitive molecule.

In an embodiment of the present disclosure, the targeting module comprises nucleic acid sequence selected from group comprising DNA, RNA and PNA or any combinations thereof, preferably a combination of DNA and RNA; and the normalizing module comprises nucleic acid sequence selected from group comprising DNA, RNA and PNA or any combinations thereof, preferably DNA.

In another embodiment of the present disclosure, the nucleic acid based sensor is for detecting target selected from group comprising Cl⁻, Ca²⁺, Mg²⁺, Zn²⁺, Cu²⁺, Fe²⁺, Pb²⁺, Cd²⁺, Hg²⁺, Ni²⁺, Co²⁺, H⁺, Na⁺, K⁺, Br⁻, I⁻, Cyanide (CN⁻), Nitrate (NO³⁻), Nitrite (NO²⁻), Nitric oxide, Phosphate (PO³⁻), Pyrophosphate (P₂O₇⁴⁻) and Reactive Oxygen species, preferably chloride (Cl⁻) ion.

In yet another embodiment of the present disclosure, the target sensitive molecule is selected from group comprising SPQ (6-methoxy-N-(3-ulphopropyl) quinolinium), MACA (10-methylacridinium-9-carboxamide), MADC (10-methylacridinium-9-N,N-dimethylcarboxamide), MANIC (N-methylacridinium-9-methylcarboxylate), DMAC (2, 10-Dimethylacridinium-9-carboxaldehyde), MAA (N-methyl-9-aminoacridinium), 6-methoxy-N-(4-sulphobutyl) quinolinium, N-dodecyl-6-methoxy-quinolinium iodide, 6-methyl-N-(3-sulphopropyl) quinolinium, 6-methoxy-N-(8-octanoic acid) quinolinium bromide, 6-methoxy-N-(8-octanoic acid) quinoliniumtetraphenyl borate, 6-methyl-N-(methyl) quinolinium bromide, 6-methyl-N-(methyl) quinolinium iodide; N, N′-dimethyl-9-9′-bisacridinium and 10,10′-Bis[3-carboxypropyl]-9,9′-biacridinium Dinitrate (BAC) or modifications and derivatives thereof, preferably 10,10′-Bis[3-carboxypropyl]-9,9′-biacridiniumDinitrate (BAC); the target insensitive fluorophore is selected from group comprising Alexafluor 568, Alexafluor 594 and Alexa 647, preferably Alexa 647; and ratio of the target sensitive molecule and the target insensitive fluorophore is 1:1.

In an embodiment of the present disclosure, the sensing module comprises sequence set forth as Seq ID No. 1; the normalizing module comprises sequence set forth as Seq ID No. 2; and the targeting module comprises sequence selected from group comprising Seq ID No. 3 and Seq ID No. 4.

In still another embodiment of the present disclosure, the aptamer targets the sensor to specific location in cell and is selected from group comprising DNA, RNA and PNA or any combinations thereof.

In still another embodiment of the present disclosure, the aptamer is RNA aptamer that specifically binds to Human Transferrin Receptor; and the RNA aptamer comprises sequence set forth as Seq ID No. 13.

In still another embodiment of the present disclosure, the sample is biological sample selected from group comprising cell, cell extract, cell lysate, tissue, tissue extract, bodily fluid, serum, blood and blood product.

The present disclosure also relates to a method of targeting nucleic acid based sensor as above, said method comprising acts of:
a) obtaining nucleic acid based sensor by method as above;
b) adding the sensor to cell for cellular uptake, to obtain a cell with the sensor; and
c) incubating the cell obtained in step b) for the nucleic acid based sensor to follow targeted cellular pathway within the cell.

In an embodiment of the present disclosure, the cell is selected from group comprising prokaryotic cell and eukaryotic cell.

In another embodiment of the present disclosure, targeting module of the nucleic acid based sensor is engineered to target the nucleic acid based sensor to follow cellular pathway within the cell.

The present disclosure also relates to a kit for obtaining or targeting nucleic acid based sensor as above or identifying and optionally quantifying target in a sample, said kit comprising components selected from group of sensing module, targeting module, normalizing module, nucleic acid based sensor, cell, sample and instructions manual or any combinations thereof.

The present disclosure also relates to a method of assembling a kit as above, said method comprising act of combining components selected from group comprising sensing module, targeting module, normalizing module, nucleic acid based sensor, cell, sample and instructions manual or any combinations thereof.

The present disclosure provides for a nucleic acid based sensor comprising sensing module, normalizing module and targeting module. The said sensor is for detecting target ions or molecules.

In an embodiment of the present disclosure, the sensing module comprises Peptide Nucleic Acid (PNA), the targeting module comprises DNA or RNA or PNA or any combinations thereof, and the normalizing module comprises DNA or RNA or PNA or any combinations thereof.

In an embodiment of the present disclosure, the sensing module of the nucleic acid based ratio-metric sensor is a peptide nucleic acid (PNA). The strand is conjugated to target sensitive molecule, for ex.: a small, fluorescent, chloride ion-sensitive molecule such as BAC (10, 10′-Bis[3-carboxypropyl]-9,9′-biacridinium Dinitrate).

In an embodiment of the present disclosure, the normalizing module is a target insensitive fluorophore, for ex.: chloride ion insensitive fluorophore such as Alexa 647, conjugated to DNA sequence that is complementary to PNA of the sensing module. Further, the targeting module is a second DNA sequence complementary to the DNA of normalizing module.

In an embodiment of the present disclosure, the sequence of the normalizing module shares partial complementarity with the sequence of the PNA strand of the sensing module. In an embodiment, the sequence of the targeting module shares partial complementarity with the sequence of the normalizing module.

The present disclosure relates to a nucleic acid based fluorescent ratio-metric sensor for detection of target. In a preferable embodiment, the present disclosure relates to a nucleic acid based fluorescent ratio-metric sensor for ion. In another embodiment, the present disclosure relates to a nucleic acid based fluorescent ratio-metric sensor for molecule.

In an embodiment, the present disclosure relates to a method to study the function and localization of any ion channel by measuring the concentration of relevant ion in a specific location within the cell, instead of measurement of ionic current. This sensor is also used to measure ion concentration in lifetime measurement mode, which is concentration independent.

In an embodiment of the present disclosure, in the presence of target, the fluorescence intensity of the target sensitive fluorophore decreases (collisional quenching). The decrease in the fluorescence intensity is the read out of the target concentration. In an embodiment of the present disclosure, the nucleic acid based fluorescent ratio-metric sensor is pH insensitive.

In an embodiment of the present disclosure, the nucleic acid based fluorescent ratio-metric sensor measures chloride ion concentration (Cl⁻), and is hereafter referred to as “Clensor” (chloride sensor) or nucleic acid based sensor.

In an embodiment of the present disclosure, the sensor module of nucleic acid based sensor is made up of PNA set forth as Seq ID No. 1 along with target sensitive molecule. The normalizing module is made up of DNA set forth as Seq ID No. 2 along with target insensitive fluorophore. The targeting module is made up of DNA set forth as Seq ID No. 3 or Seq ID No. 4.

In the present disclosure, PNA or PNA strand or PNA sequence is used interchangeably and has the same scope or meaning. In the present disclosure, DNA or DNA strand or DNA sequence is used interchangeably and has the same scope or meaning. In the present disclosure, RNA or RNA strand or RNA sequence is used interchangeably and has the same scope or meaning.

In an embodiment, the sensor of the present disclosure self assembles all its three different modules through Watson-Crick base pairing, which is stable under physiological conditions.

In an embodiment of the present disclosure, two types of targeting modules are used—A) DNA only and B) a combination of DNA and RNA. The targeting module comprising only DNA hybridizes to normalizing module to form the dsDNA domain required for intracellular targeting via ALBR The RNA sequence used in combination with DNA in the targeting module is used to achieve targeting to Transferrin pathway.

In the embodiment, DNA strand is used as normalizing module. In an embodiment of the present disclosure, the sensor has a dsDNA part (minimum 8 by sequence) resulting from the hybridization of targeting and normalizing module for the intracellular targeting. In an embodiment, the sensor comprises d(AT)4 sequence and hence is targeted to any given compartment in any cell that expresses scFv tagged protein of choice.

The present disclosure provides for measurement of Chloride ion concentration and the sequences, fluorophores etc. used to prepare the Clensor sensor molecule only by way of exemplification. The scope of the present disclosure is not limited to only the combination of particular target ion/molecule, sequences or fluorophores that make up the specific sensor molecule or to the measurement of the specific target ion/molecule. The sequences involved in the targeting, sensing or normalizing modules can be varied and conjugated depending on the requirement to different target sensitive molecules and target insensitive fluorophores to prepare various sensor molecules that measure concentration of different molecules and ions.

The present disclosure is further elaborated with the help of following examples. However, these examples should not be construed to limit the scope of the disclosure.

### EXAMPLES

### Example 1—Sensing Module

Examples 1A to 1C of the present disclosure relate to preparation of sensing module of the nucleic acid based sensor of the present disclosure. Example 1A provides for preparation of Peptide Nucleic Acid (PNA). Example 1B relates to the target sensitive fluorophore of the sensing module and Example 1C describes the characteristics of Peptide Nucleic Acid as the backbone of the sensing module.

### Example 1A—Preparation of Peptide Nucleic Acid (PNA)

In an embodiment of the present disclosure, the sensing module of the nucleic acid based ratio-metric sensor is a peptide nucleic acid (PNA). PNA strands are constructed by standard solid phase synthesis using Fmoc (Fluorenylmethyloxycarbonyl) chemistry in the laboratory. The strand is conjugated to a target sensitive molecule, for ex.: small, fluorescent, chloride ion-sensitive molecule such as BAC (10, 10′-Bis[3-carboxypropyl]-9,9′-biacridinium Dinitrate). However, other target sensitive molecule or chloride ion sensitive small molecule is also used in place of BAC as part of the sensing module.

Solid Phase PNA Synthesis using Fmoc Method:

1. Fmoc-Lys(Boc)-NovaSyn® TGA (cat. No. 04-12-2662) is weighed using a dry spatula in a dry unused vac-elute column containing a rice grain magnetic stir bar. The chunks of resin are smashed softly using spatula before weighing. The amount of resin is calculated based on loading.

NovaSyn® TGA resins are based on Tentagel, a composite of polyethylene oxide grafted on to a low cross-linked polystyrene gel-type matrix, which is amino functionalized and derivatized with the TFA-labile 4-hydroxymethylphenoxyacetic acid linker. The 90 μm beads have a narrow size distribution, excellent pressure stability and swelling properties, and high diffusion rates, making them ideally suited for both batch and continuous flow peptide synthesis. The resin swells in a wide range of solvents, enabling coupling reactions to be carried out under a variety of conditions.

2. The resin is kept for swelling in dry DCM overnight (about 6-8 hours) with gentle stirring. DCM is changed twice in between this step.

3. DCM is removed by applying vacuum, ensuring that the resin is not dried completely while applying vacuum.

4. The resin is washed with dry DCM thrice.

5. About 6004 of 20% Piperidine in dry DMF (freshly prepared in a dark bottle) is added.

The solution is gently stirred for about 30 minutes. After 30 minutes, the Piperidine solution is replaced with fresh solution and stirring is repeated for another 30 minutes.

De-protection solution: 16004, DMF+400 μL piperidine (freshly prepared in a dark bottle).

6. The piperidine solution is removed by applying vacuum.

7. The resin is washed with DCM (thrice) and DMF (twice) alternatively. Finally, the resin is washed with dry DCM thrice.

8. To this resin, about 600 μL monomer coupling solution is added.

Monomer coupling solution: Fmoc-N-A/G/C-Bhoc-COOH or Fmoc-N-T-COOH+HATU (1-[Bis(dimethylamino)methylene]-1H-1,2,3-triazolo[4,5-b]pyridinium 3-oxid hexafluorophosphate)+HOAT (1-Hydroxy-7-azabenzotriazole) in NMP (N-Methyl-2-pyrrolidone) solution.
a) PNA Monomers, HATU and HOAT are weighed in three separate tubes (1.5 mL).
b) About 150 μL of NMP is added in the tubes containing HATU and HOAT and about 300 μL of NMP is added in the tube containing monomer.
c) The tubes are vortexed.
d) About 150 μL of HATU and about 150 μL of HOAT solution is added to the tube containing monomer. The solutions are mixed by vortexing and spinning.
e) About 2 minutes before addition of the solution to the resin, about 18.2 μL of DIPEA (N,N-Diisopropylethylamine) and about 6.1 μL of Lutidine is added into the tube containing monomer, HATU and HOAT. Right after this addition, the solution turns yellow indicating the activation of monomer. If no yellow color is generated, the solution is prepared once again.

9. The solution is stirred gently and the first coupling is continued for about 4 hours. The following coupling is carried out for about 2 hours.

10. After the coupling step, the resin is washed with DCM (thrice) and DMF (twice) alternatively. Finally, the resin is washed with dry DMF thrice.

11. The cycle from step 5 onwards is repeated until the complete sequence is synthesized.

12. After coupling of the last Lysine, Fmoc is not de-protected. Mtt (methyltrityl) deprotection is carried out (for BAC conjugation) by addition of about 600 μL of a mixture of TFA:TIS:DCM=1:5:94 (freshly prepared).

13. The solution is stirred for about 5 minutes.

14. After about 5 minutes, the solution is replaced with fresh solution. After three cycles (first couple of cycles, neutralization takes place), the solution turns yellow due to free Mtt group.

Mtt deprotection solution: TFA:TIS:DCM=1:5:94 (freshly prepared).

15. The procedure is repeated till the color fades away. Finally, colorless solution indicates completion of the reaction. All the filtrate of this step is collected separately.

16. The resin is washed with DCM (thrice) and DMF (twice) alternatively. Finally, the resin is washed with dry DCM thrice.

Neutralization solution: About 10% DIPEA in DCM.

17. About 600 μL of 10% DIPEA in DCM is added for neutralization. The solution is gently stirred for about 10 minutes. After 10 minutes, the solution is replaced with fresh solution. This step is repeated thrice.

BAC NHS ester Preparation:
a) BAC=10 equivalents of resin (M. W.=654)
b) NHS (N-Hydroxysuccinimide)=4 equivalents of BAC (M.W.=115.09)
c) EDC (1-Ethyl-3-(3-dimethylaminopropyl)carbodiimide)=4 equivalents of BAC (M. W.=191.7)
d) Triethyl amine=1.8 equivalents of BAC (M.W.=101.19, 0.73g/mL)
e) DMF=About 45 mg BAC/1 mL DMF
f) 24 hour stirring at Room Temperature.

18. BAC-NHS ester (the crude mix in 10 eqv excess) is added to the resin. DIPEA is added in the solution (DIPEA:DMF=1:30). The solution is gently stirred for about 17-20 hours.

19. The resin is washed with DCM (thrice) and DMF (twice) alternatively. Finally, the resin is washed with dry DCM thrice.

20. About 600 μL of 20% piperidine in dry DMF (freshly prepared) is added. The solution is gently stirred for about 30 minutes. After 30 minutes, the piperidine solution is replaced with fresh solution and stirring is repeated for another 30 minutes.

21. The resin is washed with DCM (thrice) and DMF (twice) alternatively. Finally, the resin is washed with dry DCM thrice.

PNA cleaving solution:TFA:TIS:water=95:2.5:2.5 (freshly prepared)

22. To cleave the PNA from the resin, about 600 μL of a mixture of TFA:TIS:water=95:2.5:2.5 (freshly prepared) is added.

23. The solution is stirred vigorously for about 30 minutes. After 30 minutes, the solution is replaced with fresh solution. The procedure is repeated thrice. All the flow through is collected in a RB.

24. The cleaved resin is washed with different solvents in the following order:
a) About 600 μL Water (30 minutes, vigorous stirring)
b) About 600 μL Ethanol (30 minutes, vigorous stirring)
c) About 600 μL DCM (30 minutes, vigorous stirring)

25. All the flow through is collected in the same RB.

26. The collected solution is evaporated using a Rotavapor at about 40 to 45° C.

27. The RB is kept at about −20° C. for about 10 minutes. Dry ether is added in it and kept at about −20° C. for about 30 minutes to precipitate the synthesized PNA.

28. The supernatant is decanted carefully and MQ water is added to resuspend the precipitate.

29. The supernatant is spun at about 12000 rpm for about 10 minutes to collect any precipitate of PNA that has been removed during decanting.

30. All the solvents (except DCM and Ether) are kept in vacuum desiccators. Dry, clean long (size=20) needles and 1 mL syringes to take out solvents are used. Spatulas, needles and syringes are cleaned after every coupling and dried in hot air oven.

31. The bottle of monomer, HATU and HOAT is taken out right before the coupling step and kept at room temperature. The bottles are wiped dry and opened. The bottles are kept open for long time. Small aliquots are taken out for working stock. Unwashed/unclean spatulas are not used for weighing, to avoid contamination.

### Example 1B—Target Sensitive Molecule of Sensing Module

The sensing module of the nucleic acid based sensor comprises of Peptide Nucleic Acid conjugated to a target sensitive molecule. In an embodiment of the present disclosure, in presence of chloride ion, fluorescence of target sensitive molecule or chloride ion-sensitive molecule, such as BAC, which is conjugated to the PNA of the sensing module of the sensor, undergoes collisional quenching.

BAC fluorescence is linearly dependent on chloride ion concentration in range of about 0 to >120 mM with a Stern-Volmer quenching constant of about 36 BAC fluorescence is insensitive to physiological change in pH and to cations, non-halide anions (nitrate, phosphate, bicarbonate, sulfate) and albumin. Therefore, it is suitable for chloride ion concentration measurement in biological systems.

In an embodiment, fluorescence intensity data/Stern-Volmer plot is obtained for BAC as follows:
1. 100 mM Sodium Phosphate buffer of pH 7.4 is diluted to 10 mM working stock. The solution is filtered through 0.22 μm filter.
2. 10 μM Clensor sample is diluted to 200 nM in the filtered 10 mM Sodium Phosphate buffer of pH of 7.4 before the experiment.
3. 1 mL of 1M NaCl solution (with MQ water) is prepared in a 1.5 mL tube.
4. The cuvette is left in concentrated HCl for about 15-30 minutes before the experiment. It is washed with double distilled water properly.
5. 400 μL of 200 nM Clensor sample is taken in the cuvette.
6. Required amount of NaCl solution is added. It is mixed well using a 2-200 μL pipette. The Clensor sample is incubated for 2 minutes before data acquisition.

The fluorescence intensity is determined for fluorophores BAC, TMR and Alexa 647. The instrumental parameters used to obtain the fluorescence intensity data are provided below.

Fluoromax-4: Data Acquisition Setting:

BAC Excitation:

λex: 300-490 nm

Excitation slit width: 5 nm

λem: 505 nm

Emission slit width: 5 nm

Integration time: 0.1 sec

BAC Emission:

λex: 435 nm

Excitation slit width: 5 nm

λem: 450-550 nm

Emission slit width: 5 nm

Integration time: 0.5 sec

Alexa 647 Excitation:

λex: 500-640 nm

Excitation slit width: 2 nm

λem: 667 nm

Emission slit width: 2 nm

Integration time: 0.1 sec

Alexa 647 Emission:

λex: 650 nm

Excitation slit width: 2 nm

λem: 655-700 nm

Emission slit width: 2 nm

Integration time: 0.1 sec

TMR Excitation:

λex: 400-600 nm

Excitation slit width: 2 nm

λem: 575 nm

Emission slit width: 2 nm

Integration time: 0.1 sec

TMR Emission:

λex: 540 nm

Excitation slit width: 3 nm

λem: 550-600 nm

Emission slit width: 3 nm

Integration time: 0.1 sec

For data analysis, emission is considered at 505 nm for BAC (G), emission at 670 nm for Alexa 647 (R) and emission at 570 nm for TMR (R). Ratio of R to G is taken without a dilution correction. Blank subtraction is performed for each wavelength.

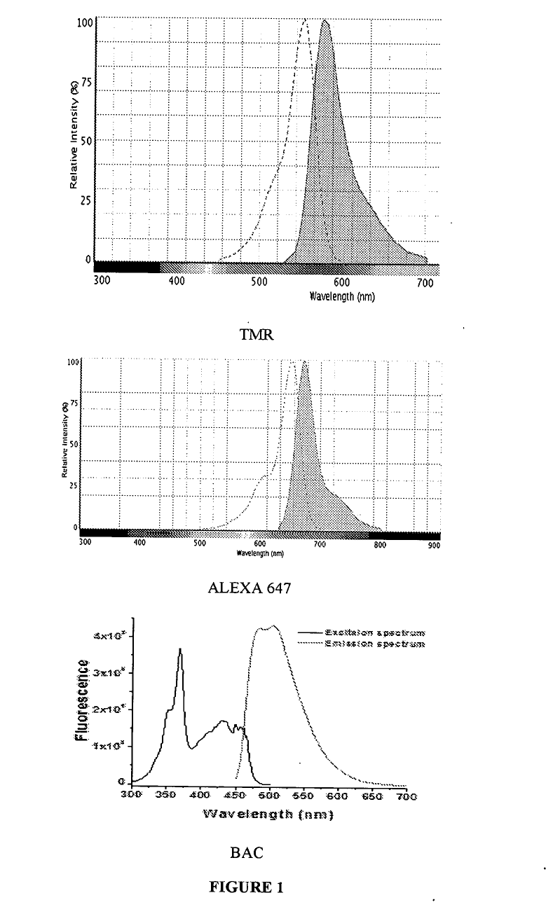

FIG. 1 of the present disclosure depicts excitation and emission spectra of BAC, TMR and Alexa 647 fluorophores. It is derived from the excitation and emission spectra shown in FIG. 1 that spectral overlap between Alexa 647 and BAC is negligible compared to spectral overlap between TMA and BAC. This is very important to avoid any crosstalk between target or chloride sensitive molecule and insensitive fluorophores during microscopy imaging.

### Example 1C—PNA as Backbone of Sensing Module

In an embodiment, BAC conjugated to DNA sequence shows less sensitivity to chloride compared to free BAC. This is primarily due to electrostatic effect of negatively charged DNA backbone.

In an embodiment of the present disclosure, chloride sensitivity is determined for different nucleic acid probes, comprising BAC conjugated to ssDNA, dsDNA, ssPNA, dsPNA etc. It is observed that neutral backbone of PNA as sensing module leads to higher sensitivity compared to negatively charged DNA backbone. Hence, in an embodiment of the present disclosure, to improve the sensitivity, Peptide Nucleic Acid (PNA) strand is used in the sensing module.

FIG. 3 of the present disclosure provides a tabular and pictorial representation showing the effect of negatively charged backbone of DNA on the chloride sensitivity. DNA strands and PNA strands are shown as black and pink lines respectively. In FIG. 3, the ratio of fluorescence intensity of the chloride sensitive and insensitive fluorophores at various chloride concentrations are plotted as a function of chloride concentration. This is similar to Stern-Volmer plot (F₀/F=1+K_{SV}*[Q], where F₀=Fluorescence intensity in the absence of quencher, F=Fluorescence intensity in the presence of quencher, K_{SV}=Stern-Volmer constant and [Q]=Quencher concentration). Higher slope of the linear plot and fold change indicates the higher sensitivity of the fluorophore to the quencher (chloride).

Thus, it is derived from this example that BAC is suited as a target sensitive molecule for Clensor.

### Example 2—Target Sensitive Molecule and Target Insensitive Fluorophore

The nucleic acid based sensor for detecting target, comprises of sensing module, normalizing module and targeting module. Provided below is a list of target sensitive molecules or chloride sensitive fluorophores which are used in the sensing module of the sensor of the present disclosure.

TABLE 1

Lucigenin-N,N′-dimethyl-9,9′bisacridinium.
SPQ-6-Methoxy-N-(3-sulfopropyl)-quinolinium.
MACA-10-Methylacridinium-9-carboxamide.
MADC-10-Methylacridinium-9-N,N′dimethylcarboxamide.
MAMC-N-Methylacridinium-9-methylcarboxylate.
DMAC-2,10-Dimethylacridinium-9-carboxaldehyde.
MAA-N-Methyl-9-aminoacridinium iodide.
6-Methoxy-N-(4-sulfobutyl)-quinolinium.
N-dodecyl 6-Methoxy quinolinium iodide.
6-Methyl-N-(3-sulfopropyl)-quinolinium.
6-Methoxy-N-(8 octanoic acid) quinolinium bromide.
6-Methoxy-N-(8 octanoic acid) tetraphenyl borate.
6-Methyl-N-(methyl) quinolinium bromide.
6-Methyl-N-(methyl) quinolinium iodide.
SPA-N (sulphopropyl) acridinium.
3-(10-methylacridinium-9-yl) Propionic acid tetrafluoroboron.

In an embodiment of the present disclosure, the normalizing module of the sensor is target insensitive fluorophore or chloride ion insensitive fluorophore such as Alexa 647 conjugated to DNA sequence that is complementary to PNA of sensing module. Alternatively chloride insensitive fluorophores such as Alexa fluor 568, Alexa fluor 594, Alexa fluor 647 etc. are also used.

### Example 3—Clensor

In an embodiment of the present disclosure, the method of obtaining nucleic acid based sensor or Clensor, comprising DNA strand D1 as targeting module, D2 as normalizing module and PNA strand P as sensing module is broadly as follows:
1. The DNA strands (D1 and D2) are obtained from IBA GmbH, Germany.
2. PNA strands (P) are synthesized as per the procedure described in Example 1A.
3. BAC is synthesized following protocol known in the art.
4. BAC is chemically conjugated to PNA strand (P).
5. BAC conjugated PNA (BAC-PNA) is purified by HPLC.
6. D1, D2 and BAC-PNA are dissolved in Milli Q water.
7. Concentration of each strand is determined using UV-Vis absorbance.
8. D1, D2 and BAC-PNA are mixed in about 10 mM phosphate buffer of pH of about 7.4 in 1:1:1 ratio to prepare nucleic acid based sensor or Clensor sample of final concentration of about 10 μM.

As an exemplification, sequences of D1, D2 and PNA (P) are described in below Table 2 of the present disclosure.

TABLE 2

| Seq ID | | |
|---|---|---|
| No./Module | Name | Sequence |
| Seq ID No. 1 | PNA strand (P) | NH_{ε}-Lys-ATC AAC ACT GCA-Lys-COOH |
| Module | Sensing module | Chloride sensitive molecule |
| | | (BAC) + Seq ID No. 1 |
| Seq ID No. 2 | DNA strand (D2) | 5′ TATA [symbol] ATA GGATCTTGCTGTCTGGTG |
| | | TGC AGT GTT GAT 3′ (internal |
| | | Alexa 647 modification on |
| | | the T shown in bold and italics) |
| Module | Normalizing | Chloride insensitive fluorophore |
| | module | (Alexa 647) + Seq ID No. 2 |
| Seq ID No. 3 | DNA strand (D1) | 5′ CACCAGACAGCAAGATCC TATATATA 3′ |
| Module | Targeting module | Seq ID No. 3 |
| Seq ID No. 4 | DNA RNA hybrid | 5′CACCAGACAGCAAGATCCTATATATAGGGGGA |
| | strand (D1 Tfapt) | [symbol] AA [symbol] AAGGGA [symbol] GGAAA [symbol] G [symbol] A |
| | | [symbol] 3′ |
| | | This a 69 mer RNA-DNA hybrid sequence. |
| | | 26 bases from 5′ end are DNA bases |
| | | (shown in red). Remaining bases (43 |
| | | bases) shown in black are RNA bases. |
| | | Bases shown in bold and italics are |
| | | 2′ fluoro modified C and U. |
| Module | Targeting module | Seq ID No. 4 |
| | with RNA aptamer | |
| | against human | |
| | transferrin | |
| | receptor | |
| Seq ID No. 13 | RNA Aptamer | GGGGGA [symbol] CAA [symbol] AAGGGA [symbol] GGAAA [symbol] G [symbol] |
| | | [symbol] A [symbol] A [symbol] |

Any PNA sequence that forms a stable duplex with the complementary DNA strand (D2) of the sensor molecule at physiological condition is also used in the sensor molecule of the present disclosure.

### Example 4—Clensor and Clensor^{Tf}

The targeting module comprises nucleic acid sequence complementary to nucleic acid sequence of the normalizing module, optionally with an aptamer. The SEQ ID No. 4 is DNA-RNA hybrid strand (D1Tfapt) Targeting module with RNA aptamer against human transferrin receptor.

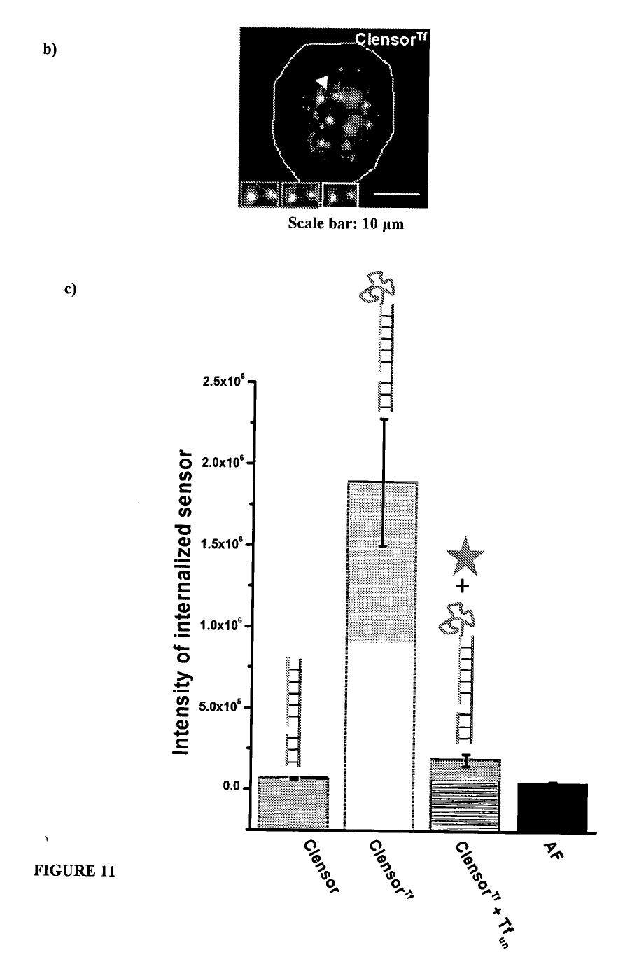

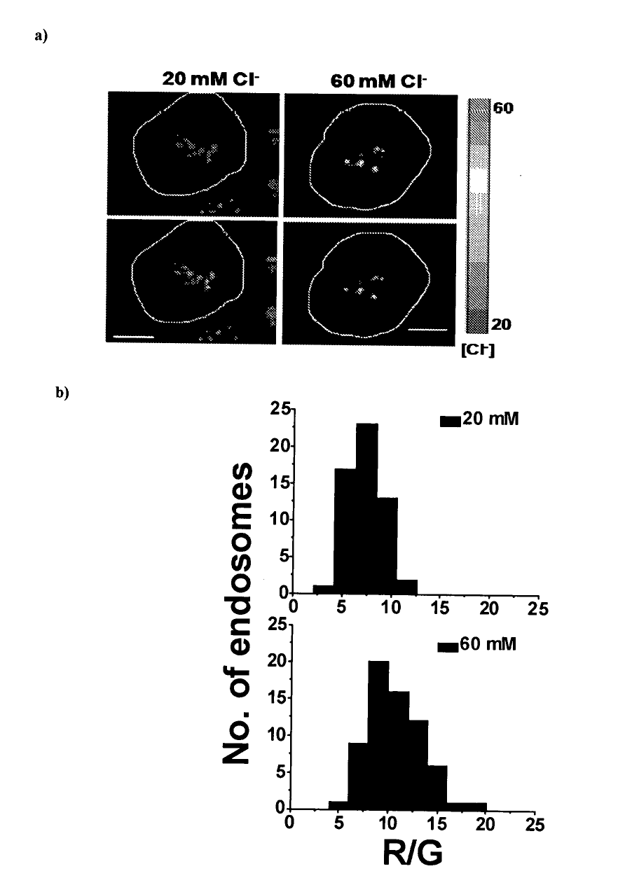

FIG. 11a of the present disclosure describes the design of Clensor^{Tf}. The pink line represents the sensing module comprising the PNA conjugated to the chloride sensitive fluorophore BAC represented by the green filled in star. The yellow line represents the targeting module D1, conjugated to Transferrin aptamer represented by the blue line. The brown line represents normalizing module D1 conjugated to a chloride insensitive fluorophore represented by the red filled in circle.

Nucleic acid based sensor preparation: Stock solutions of PNA and DNA are made in MQ water and stored at −20° C. Stock solution of 100 mM Sodium Phosphate buffer of pH of 7.4 and 500 mM EDTA of pH of 8.0 are prepared and filtered using 0.22 μm disk filters. For a specific Nucleic acid based sensor, all the relevant component strands are mixed in equimolar ratio at a final concentration of 10 μM (It has been observed that at 30 μM stand concentration, the Nucleic acid based sensor starts forming aggregates over long period of storage. So, it is advisable to make the Nucleic acid based sensor at a lower concentration) in 10 mM Sodium Phosphate buffer of pH of 7.4 (no extra salt added). Annealing is done by heating the solution at 90° C. for 5 min and cooling at the rate of 5° C./15 min. All the nucleic acid based sensors are incubated at 4° C. at least for 48 hours before experiments.

Before pulsing nucleic acid based sensor to cells, the nucleic acid based sensors are diluted in 1×M1 buffer+1 mg/mL BSA+2 mg/mL D-glucose to appropriate concentration. This preparation method is applicable for Clensor and all other sensors based on nucleic acid scaffolds during optimization of the sensor. Stock solutions of DNA-RNA hybrid are made in MQ water or nuclease free water, used for RNA work in the lab and stored at −80° C. in small aliquots (amount required for one 100 μL of sensor). Frequent freeze thaw cycles are avoided.

For Clensor^{Tf} sample, all the relevant component strands are mixed in equimolar ratio at a final concentration of 10 μM (It has been observed that at 30 μM stand concentration, the nucleic acid based sensor starts forming aggregates over long period of storage. So, it is advisable to make the nucleic acid based sensors at a lower concentration) in 10 mM

Sodium Phosphate buffer of pH of 7.4 and 1 mM EDTA of pH of 8 (no extra salt added). Annealing is done by heating the solution at 90° C. for 5 min and cooling at a rate of 5° C./15 min. All the nucleic acid based sensors are incubated at 4° C. at least for 48 hours before experiments. Before pulsing cells with Clensor^{Tf}, the sensor is diluted in 1×M1 buffer+1.5 mg/mL BSA+2 mg/mL D-glucose to appropriate concentration and incubated for 30 minutes at room temperature for proper folding of RNA aptamer against transferrin.

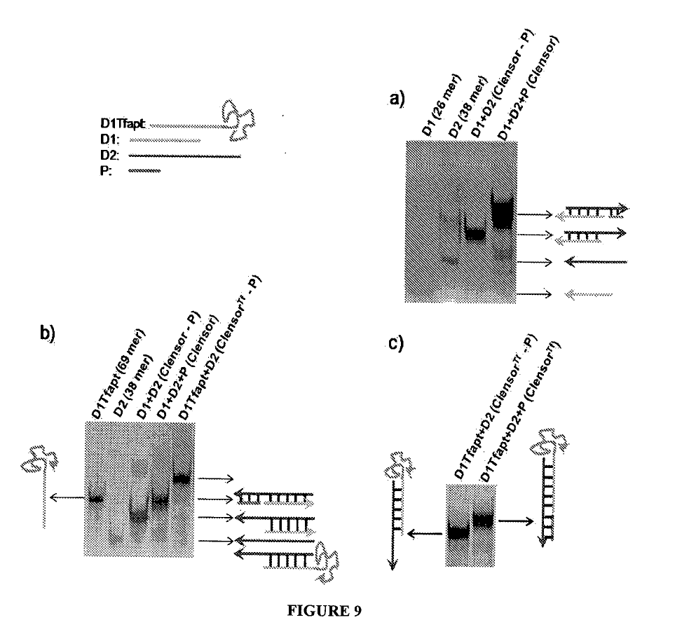

FIG. 9 of the present disclosure depicts a Gel Mobility Shift Assay for formation of Clensor and Clensor^{Tf}. It is derived from the Gel Mobility Shift Assay that three different modules combine to form Clensor. Gel mobility shift assay is a molecular proof of the sensor formation.

The table below provides sequences of all DNA sequences used during optimization of the sensor.

TABLE 3

| Sequences | Seq ID No. |
|---|---|
| CCCTAACCCTAACCCTAACCCGACTCACTGTTTGTCTGTCGTTCTAGGATATATATTT | Seq ID No. 5 |
| ATATATATCCTAGAACGACAGACAAACAAGTGAGTCTTTGTTATGTGTTATGTGTTAT | Seq ID No. 6 |
| AAATATATATCCTAGAACGACAGACAAACAGTGAGTCTTTGTTATGTGTTATGTGTTAT | Seq ID No. 7 |
| CCCTAACCCTAACCCTAACCCGACTCACTGTTTGTCTGTCGTTC [symbol] CGGATATATAT | Seq ID No. 8 |
| ATATATATCCGAGAACGACAGACAAACAGTGAGTCTTTGTTATGTGTTATGTGTTAT | Seq ID No. 9 |
| ATATATATCCGGAACGACAGACAAACAGTGAGTCTTTGTTATGTGTTATGTGTTAT | Seq ID No. 10 |
| ATATATATCCGAACGACAGACAAACAGTGAGTCTTTGTTATGTGTTATGTGTTAT | Seq ID No. 11 |
| ATATATATCCAACGACAGACAAACAGTGAGTCTTTGTTATGTGTTATGTGTTAT | Seq ID No. 12 |

In the table above, the DNA sequences which are used to optimize Clensor are depicted. From the sequences depicted above, some sequences are used as sensing modules and some sequences as normalizing modules during optimization of the sensor molecule. In the table depicted above, the sequences in same colors are complementary. In each sequence, the part of the sequence depicted in red is required for the general targeting strategy of the sensor to a specific location. The black bases of the sequences are the unpaired bases. The part of the sequence depicted in blue forms mismatched duplex at neutral pH and forms i-motif at acidic pH.

Upon measuring the chloride sensitivity of each of these sequences Seq ID Nos. 7-12, it is observed that BAC (chloride sensitive fluorophore) conjugated to DNA shows very low chloride sensitivity compared to free BAC and BAC conjugated to PNA (FIG. 3 and Example 1C). Hence, a PNA sequence that shows thermal stability upon hybridization to complementary DNA strand is chosen to prepare the sensor module of Clensor.

### Example 5—Ratiometric Ionic Concentration Measurement Using Clensor

In an embodiment, the present disclosure relates to a fluorescent ratio-metric sensor for quantitative chloride ion measurement at specific location inside the cell. Fluorescent measurements are sensitive to uneven dye loading, leakage of dye, and photo-bleaching, as well as unequal cell thickness. Ratiometric imaging considerably reduces these effects.

In an embodiment of the present disclosure, to achieve ratio-metric sensing, target/chloride ion insensitive fluorophore, such as Alexa 647 is conjugated to the DNA sequence that is complementary to PNA of sensing module. This is the normalizing module. The target insensitive fluorophore such as Alexa 647 chromophore is not sensitive to pH or chloride concentrations. Therefore, the ratio of target insensitive fluorophore to target sensitive molecule or Alexa 647 fluorescence (red) to BAC fluorescence (green) increases linearly with chloride ion concentration from about 0 to about 200 mM chloride ion.

In an embodiment of the present disclosure, an in vitro experiment is performed to check the linear regime of chloride ion sensitivity of Clensor. This ratio-metric chloride ion sensing mechanism of Clensor enables quantitative measurement in complex biological systems.

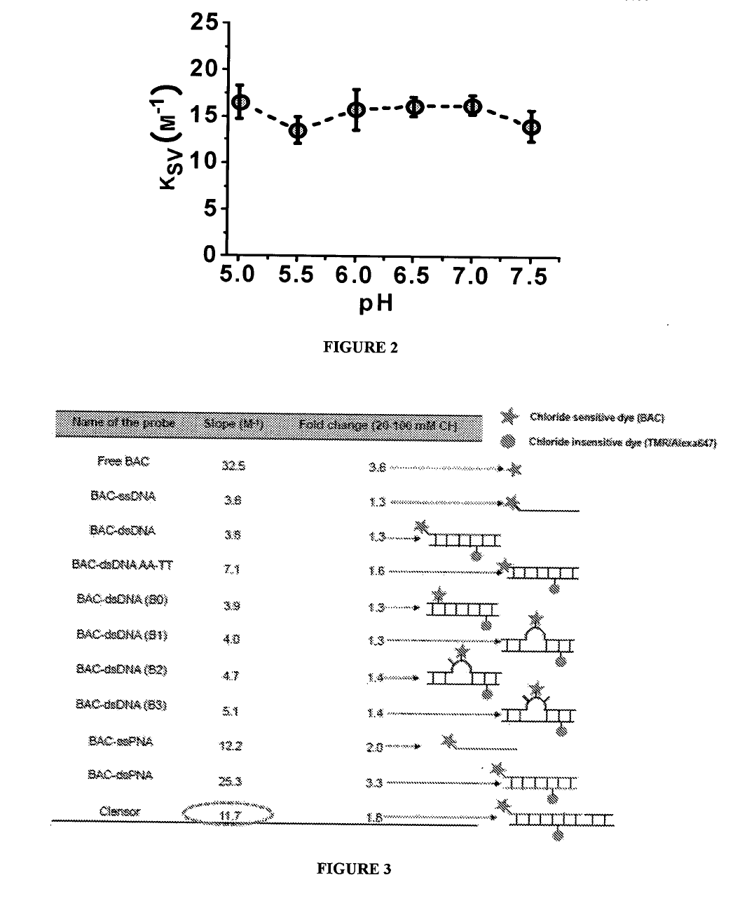

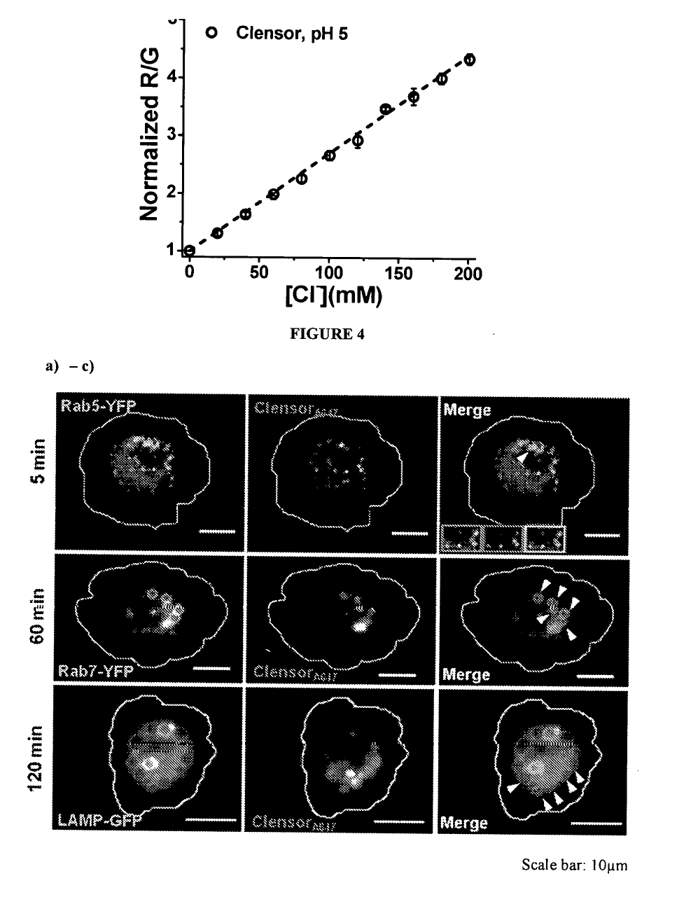

FIG. 2 of the present disclosure depicts a plot showing chloride sensitivity of Clensor at different physiological pH values. FIG. 4 of the present disclosure depicts the plot showing the linear range of about 0-200 mM of chloride sensitivity of Clensor at low pH (pH of about 5.0). This property of Clensor enables quantitative measurement of chloride concentration in lysosome.

The difference in the nature of Clensor and BAC-TMR-Dextran, known for ratiometric imaging for chloride ion measurement is derived from FIG. 7. In FIG. 7a), specific hybridization between strand P (shown as pink line) and strand D2 (shown as black line) confirms a fixed 1:1 ratio of BAC (shown as green star) and Alexa 647 (shown as red filled circle). Therefore, in a Clensor sample, all the species are molecularly identical.

In FIG. 7b), schematic representation of different molecularly non-identical species available in a BAC-TMR-Dextran sample is shown. Dextran (shown as gray filled circle), functional groups (shown as black lines), BAC (shown as green star) and TMR is shown as red filled circle. Typically, Dextran conjugates are prepared by reacting water-soluble amino Dextran with the succinimidyl ester derivatives of the appropriate dyes (BAC or TMR). As there are multiple functional groups available for fluorophore conjugation on a single Dextran, the degree of labeling is difficult to control. Therefore, in a sample of BAC-TMR-Dextran, there are species which are not molecularly identical (possibilities are shown in FIG. 7b).

Fluorescence quenching is usually monitored by the loss of fluorescence intensity. (It is also monitored by lifetime measurement which is concentration independent.) Loss of fluorescence intensity could also be due to less dye loading or photobleaching of the fluorophore other than quenching. Therefore, determination of chloride ion concentration using ratio-metric approach is more reliable and quantitative. Specific hybridization between strand P and strand D2 of the sensor molecule confirms a fixed 1:1 ratio of BAC and Alexa 647. Therefore, in a Clensor sample, all the species are molecularly identical and provides a narrow distribution of R/G ratio. Therefore, any spread in the distribution reports on the biological spread of the chloride ion concentration reliably. On the other hand, there are different molecularly non-identical species available in a BAC-TMR-dextran sample. This leads to a broad distribution of the R/G ratio. Therefore, BAC-TMR-dextran is not able to report on the biological spread of chloride ion concentration reliably.

In Clensor, a fixed 1:1 ratio of BAC and Alexa 647 is achieved by virtue of specific nucleic acid strand hybridization. Clensor uses Alexa 647 as normalizing fluorophore, which shows insignificant spectral overlap with BAC.

### Example 6—Measurement of Chloride Concentration and pH in Compartments of Drosophila Hemocytes

In this experiment, the nucleic acid based sensor (Clensor) is targeted to early endosomes (EE), late endosomes (LE) and lysosomes (LY) in Drosophila Hemocytes and Chloride concentration and pH is measured in these compartments.

Hemocytes used in the present disclosure are isolated from flies of different genotype. The fly stocks and their sources are provided in table 4 below.

TABLE 4

| Genotype | Reference |
|---|---|
| CS | Bloomington Drosophila Stock Center at Indian University |
| y [symbol] w [symbol] ; P{EP}ClC-b [symbol] | Bloomington Drosophila Stock Center at Indian University |
| y [symbol] v [symbol] ; P{TRiPJF01844}attP2 | Bloomington Drosophila Stock Center at Indian University |
| y [symbol] w [symbol] ; P{EP}ClC-c [symbol] | Bloomington Drosophila Stock Center at Indian University |
| y [symbol] v [symbol] ; P{TRiPJF02360}attP2 | Bloomington Drosophila Stock Center at Indian University |
| w [symbol] ; P{Cg-GAL4A}2 | Bloomington Drosophila Stock Center at Indian University |
| y [symbol] w [symbol] ; P{UASp-YFP.Rab5}02 | Bloomington Drosophila Stock Center at Indian University |
| y [symbol] w [symbol] ; P{UASp-YFP.Rab7}21/SM5 | Bloomington Drosophila Stock Center at Indian University |
| W [symbol] ; P[w [symbol] , uasGFP-LAMP] [symbol] /Cyo; | Agit from H. Krämer (University of Texas Southwestern Medical |
| TM6b, Hu boss/Sb boss [symbol] | Center, Dallas, TX) J. Cell Sci. 2005, 118, 3663-3673. |

[symbol]

indicates data missing or illegible when filed

In an embodiment of the present disclosure, for intracellular targeting of the sensor to a given compartment along a specific pathway, a dsDNA domain is incorporated. The dsDNA domain is represented by combination of targeting module and normalizing module. It facilitates chloride ion measurement in cellular microenvironment of any cell type. To achieve this, a second DNA sequence complementary to the DNA of normalizing module is used. This is the targeting module. In an embodiment, the dsDNA domain also acts as a negatively charged ligand for a set of cell surface receptors called Anionic Ligand Binding Receptors (ALBR) and enables targeting of Clensor in endosomal-lysosomal pathway of Drosophila hemocytes.

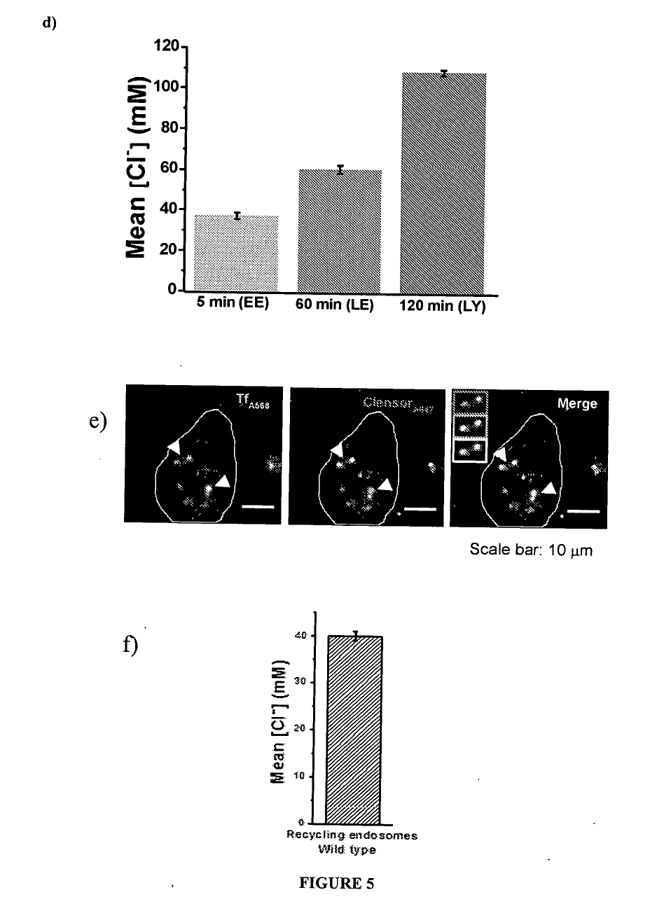

ALBR (Anionic Ligand Binding Receptors) expressed on the surface of Drosophila hemocytes specifically binds the double stranded region of Clensor when the cells are incubated in a solution of Clensor (pulse). Upon binding, the receptor ligand complex undergoes endocytosis. They reach early endosomes, late endosome and lysosome as a function of time (chase). Colocalization with endosomal protein markers such as Rab 5 (EE marker), Rab 7 (LE and LY marker) and LAMP1 (LY marker) confirms the specific targeting, as seen in FIG. 5 of the present disclosure.

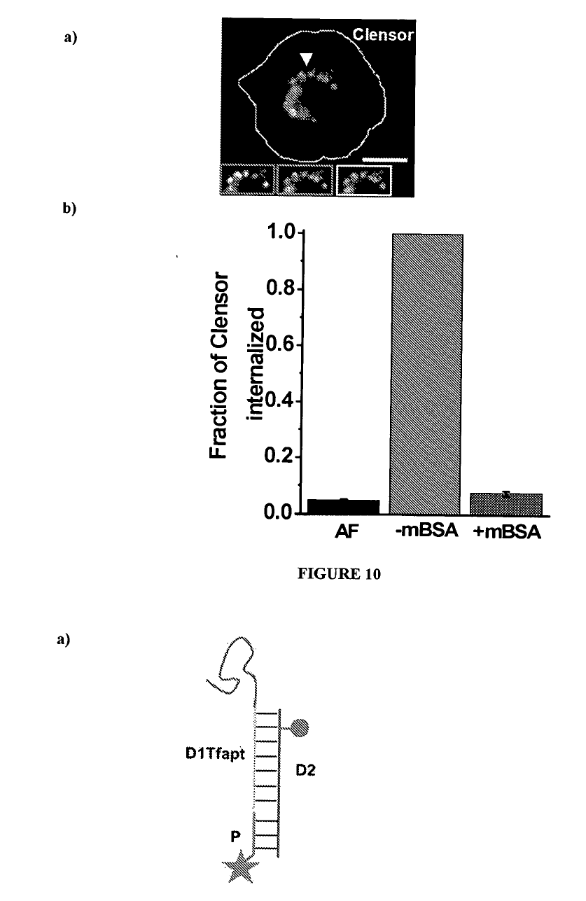

FIG. 10a of the present disclosure depicts the representative image of Drosophila Hemocytes pulsed with Clensor.

FIG. 15 of the present disclosure depicts percentage co-localization of Clensor with different endosomal markers at given time points. It is derived from the graph of FIG. 15 that Clensor maximally localizes within early endosomes, late endosomes and lysosomes at 5 min, 60 min and 120 min respectively.

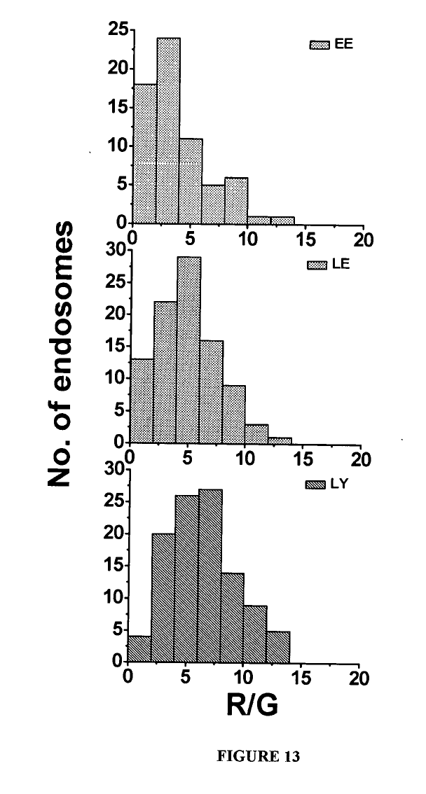

FIG. 13 of the present disclosure depicts Histogram showing the shift in R/G ratios of endosomes undergoing maturation. It is observed from the figure that Clensor reports spatiotemporal change in Chloride ion concentration during endosomal maturation with accuracy. It is derived from the figure that the RIG ratio of endosomes in live cells increases as a function of time indicating the increase in chloride concentration in respective compartments associated with endosomal maturation.

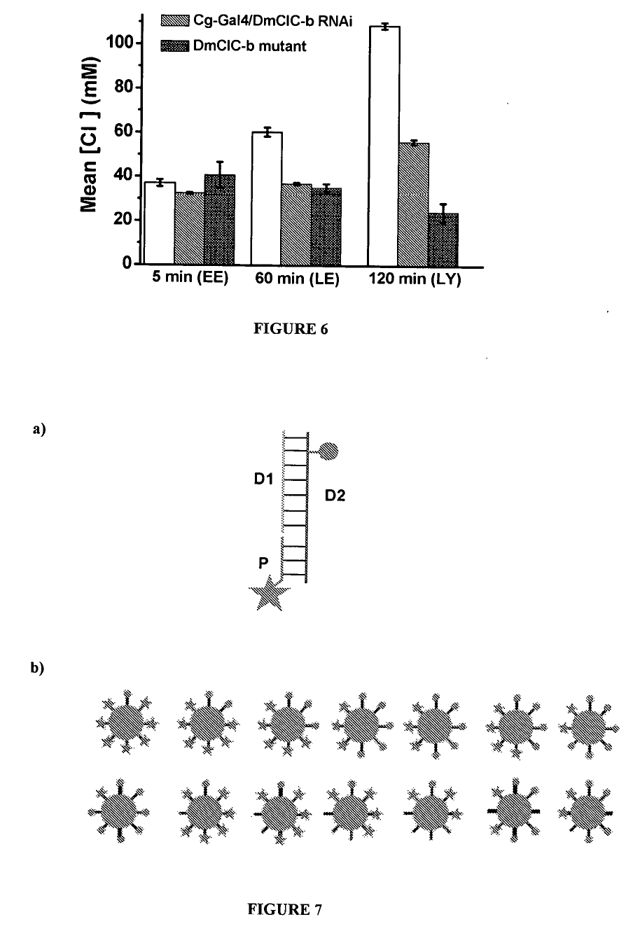

In an embodiment of the present disclosure, Clensor is used to study function and localization of ClC family, putative intracellular chloride transporters in Drosophila (DmClC) within endosomes along the ALBR pathway, as seen in FIG. 6 of the present disclosure. The DmClC-b and DmClC-c chloride channels are studied in an embodiment of the present disclosure.

Two genetic backgrounds that perturb one of these genes, that is DmClC-b, are chosen: an RNAi knockdown (Cg-Ga14/DmClCb RNAi) and a mutant generated by EP element insertion in the intron of the gene. Live cell chloride measurement in these genetic backgrounds indicates that DmClC-b resides on the late endosome and lysosomes and helps in chloride accumulation in these compartments. The mutant fly lines are obtained from Bloomington Drosophila Stock Center at Indiana University.

In this example, DmClC-b is disrupted by RNAi knockdown and insertional mutagenesis. Chloride ion concentration measurements in such cells show impaired Chloride ion accumulation specifically in late endosome and lysosomes of Drosophila hemocytes (FIG. 6). Further, pH measurements show defective acidification in these compartments compared to wild type cells. This indicates that DmClC-b localizes on late endosome and lysosome and facilitates acidification in late endosomes and lysosomes via Chloride ion accumulation.

As seen above and in Table 9, pH measurements made in these compartments indicate that chloride accumulation in these compartments facilitates acidification of these compartments.

Protocol for pH Measurement in Hemocytes I Switch Sensor Preparation:

All oligonucleotides are ethanol precipitated and quantified by their ultraviolet absorbance. An I switch sensor is made by mixing the DNA oligonucleotides O1, O2 and O3 (represented by Seq ID Nos.14-16 in Table 5) in equimolar ratios, heating at 90° C. for 5 minutes, and then slowly cooling to room temperature at 5° C. per 15 min.

TABLE 5

| Oligo- | | Seq |
|---|---|---|
| nucleo- | | ID |
| tide | Sequences | Nos. |
| O1 | 5′-Alexa-488- | Seq ID |
| | CCCCAACCCCAATACATTTTACGCCTGGTG | No. 14 |
| | CC-3′ | |
| O2 | 5′-CCGACCGCAGGATCCTATAAAACCCCA | Seq ID |
| | ACCCC-Alexa-647-3′ | No. 15 |
| O3 | 5′- | Seq ID |
| | TTATAGGATCCTGCGGTCGGAGGCACCAGG | No. 16 |
| | CGTAAAATGTA-3′ | |

The sequences in same color are self-complementary. Red nucleotide is unpaired nucleotide.

This preparation is carried out with oligonucleotide concentrations of 5 μM, in 10 mM phosphate buffer of pH of 5.5, in the presence of 100 mM KCl. The I switch sensors are then equilibrated at 4° C. overnight. Fluorescently labeled I switch sensor is prepared in a similar manner with fluorophore-labeled oligonucleotides. The I switch sensors are used within 7 days of annealing.

pH Measurements with I Switch Sensor:

Hemocytes are washed with 1×M1 buffer (150 mM NaCl, 5 mM KCl, 1 mM CaCl₂, 1 mM MgCl₂, 20 mM HEPES pH 7.2) prior to labeling. Hemocytes are incubated with 500 nM of I switch sensor (diluted in 1×M1 buffer for about 5-10 minutes (depending on the signal again, *for pH measurements in early endosomes 5 min pulse is given*) and then washed 3-4 times with 1×M1 and then chased for an additional 5 to 120 minutes. For a chase longer than 5 minutes, 1×M1 is replaced by complete insect medium (Gibco, Invitrogen) and transferred to a 20° C. incubator. For 5 minute chases, cells are washed and then imaged in 1×M1.

For pH measurement experiments, after pulsing with I switch sensor, cells are chased for (i) 5 minutes, (ii) 60 minutes and (iii) 120 minutes. Each set of cells are then imaged live, acquiring three images for each set of cells (i) by exciting donor (488 nm) (ii) by exciting acceptor (630 nm) and (iii) the FRET image (exciting at 488 nm and acquiring at 647 nm).

The intracellular pH standard curve is obtained by addition of 500 nM I switch sensor, incubation for 30 minutes (for development of a signal), washing 3-4 times with 1×M1 followed by a 5 minute chase in same medium. Hemocytes are then briefly fixed with about 200 μL 2.5% Paraformaldehyde (PFA) for about 2 minutes, quickly washed 3 times and retained in 1×M. An ionophore, Nigericin, is diluted to 10 μM with clamping buffer (150 mM KCl, 5 mM NaCl, 1 mM CaCl₂, 1 mM MgCl₂, 20 mM HEPES, pH 5.0 to 7.0) of desired pH (ranging from about 5-7). 1000 mL of this clamping buffer is added to the previously fixed cells, incubated for 30 minutes and then imaged in the same buffer. Table 6 of the present disclosure depicts a tabular representation of the results obtained during the pH measurement in early endosome, late endosome and lysosome of Hemocytes of indicated genotypes.

TABLE 6

| | CS Hemocytes (pH) (Canton Specia - wild | DmClC-b mutant |
|---|---|---|
| Time (min) | type strain of Drosophila melanogaster) | Hemocytes (pH) |
| 5 (EE) | 5.8 ± 0.18 | 5.8 ± 0.18 |
| 60 (LE) | 5.4 ± 0.08 | 5.7 ± 0.06 |
| 120 (LY) | 5.2 ± 0.19 | 5.7 ± 0.05 |

This table indicates that, pH is disrupted in the mutant genotype compared to the wild type, which shows that Chloride ion concentration has a role to play in maintaining the pH of the indicated compartments.

Chloride Concentration Measurements in Hemocytes with Clensor:

Hemocytes are washed with 1×M1 buffer (150 mM NaCl, 5 m1\4 KCl, 1 mM CaCl₂, 1 mM MgCl₂, 20 mM HEPES pH 7.2) prior to labeling. Hemocytes are incubated with 2 μM of Clensor sample (diluted in 1×M1 buffer) for 5-10 minutes (depending on the signal again, *for measurements in early endosomes 5 min pulse is given*) and then washed 3-4 times with 1×M1 and then chased for an additional 5 to 120 minutes. For a chase longer than 5 minutes, 1×M1 is replaced by complete insect medium and transferred to a 20° C. incubator. For 5 minute chases, cells are washed and then imaged in 1×M1.

For Chloride ion (Cl³¹ ) measurement experiments, after pulsing with Clensor, cells are chased for (i) 5 min (Early Endosome), (ii) 60 min (Late endosome) and (iii) 120 min (Lysosome). Each set of cells are then imaged live, acquiring two images for each set of cells. (i) Ex=480/20, Dichroic=500-600 BP, Em=535/40 and (ii) Ex=640/30, Dichroic=665 LP, Em=690/50.

Chloride Clamping:

Hemocytes are pulsed with 2 μM of Clensor sample for 30 minutes (for development of a signal), washed 3-4 times with 1×M1 followed by a 5 minute chase in same medium. Cells are then fixed with 200 μL 2.5% paraformaldehyde (PFA) for 20 minutes at room temperature, quickly washed 3 times and retained in 1×M1. To obtain intracellular Chloride ion (Cl⁻) calibration curve, perfusate and endosomal Chloride ion (Cl⁻) concentration are equalized by incubating the previously fixed cells in 120 mM KCl/KNO₃, 20 mM NaCl/NaNO₃, 1 mM CaCl₂/Ca(NO₃)₂, 1 mM MgCl₂/Mg(NO₃)₂, and 10 mM HEPES at pH 7.2, with Chloride ion (Cl⁻) concentration from about 0-80 mM (NO₃⁻ replacing Cl⁻) for about 3 hours at room temperature. Chloride clamping solution contains Nigericin (10 μM)+Valinomycin (10 μM)+CCCP (5 μM)+Monensin (10 μM)+Bafilomycin (200 nM). The cells are imaged in the same buffer.

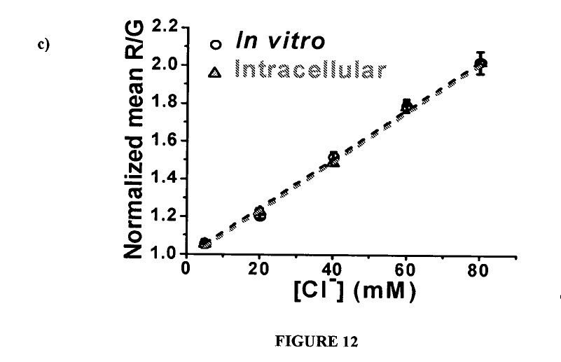

FIG. 12 of the present disclosure provides the quantitative performance of Clensor within the endosomes of Drosophila hemocytes. FIG. 12a of the present disclosure depicts clamping hemocytes (pulsed with Clensor) at known concentrations of chloride. These are Alexa 647 channel and respective pseudocolour R/G map of hemocytes pulsed with Clensor and clamped at 20 mM and 60 mM Chloride concentration (Scale bar: 10 μm). These representative images indicate the change in R/G ratio within the endosomes of the chloride clamped hemocytes as a function of chloride concentration in the extracellular buffer used for chloride clamping. FIG. 12b provides a Histogram showing R/G ratios of endosomes when clamped at different chloride concentrations. These Histograms show spread of R/G ratios of endosomes clamped at 5 mM (green) and 60 mM (red) Chloride concentration. This indicates that R/G ratio increases as a function of chloride concentration in the clamping buffer. FIG. 12c depicts the in vitro and intracellular performance (chloride calibration profile) for Clensor.

### Example 7-Clensor^{Tf} in Recycling Endosomes of S2R+ Cells

Further, in an embodiment of the present disclosure, the targeting module of the sensor molecule is modified by addition of an aptamer against Human Transferrin Receptor at the 3′ end, as seen in FIG. 5e of the present disclosure. In a particular embodiment of the present disclosure, the nucleic acid based sensor or chloride sensor (Clensor) containing D1Tfapt as targeting module (Clensor^{Tf}) is used to target the sensor in recycling endosomes of Drosophila S2R+ cells (FIGS. 5e and 5f).

Drosophila S2R+ cells are obtained as a gift from Dr Satyajit Mayor, NCBS. The name of the cells line is Schneider's Drosophila Line 2 [D. Mel. (2), SL2] (ATCC® CRL-1963™.

It is possible to modify the targeting module with an aptamer, against a particular cell surface receptor. The sensor acts as a natural ligand for the receptor. It is internalized in cells that express that receptor and follows the intracellular trafficking pathway of the endogenous ligand. Hence, it enables probing the intracellular ionic environment in that pathway. This modified sensor is named as Clensor^{Tf}. Clensor^{Tf} is targeted specifically to Recycling Endosomes (RE). Co-localization of Transferrin, a recycling endosomal marker and Clensor^{Tf} confirms that Clensor^{Tf} is specifically targeted to recycling endosomes.

The targeting module of the sensor is modified with an RNA aptamer (D1Tfapt) that specifically binds Human Transferrin Receptor and acts as its cognate ligand. The sensor is named as Clensor^{Tf}. Drosophila S2R+ cells stably express the Human Transferrin Receptor. Therefore, during the incubation of S2R+ cells with Clensor^{Tf}, the aptamer binds Human Transferrin Receptor on the cell surface. Upon internalization, Clensor^{Tf} reaches Recycling Endosome as function of time. Colocalization of Clensor^{Tf} with recycling endosomal marker protein, Transferrin, confirms that Clensor^{Tf} resides in recycling endosomes. Table 7 of the present disclosure provides different time durations of pulse and chase used to measure Chloride Concentration and pH in different endosomal compartments.

TABLE 7

| Target | | |
|---|---|---|
| Compartment | Pulse time | Chase time |
| Early Endosome | 5 minutes at room | NA |
| | temperature | |
| Late Endosome | 10 minutes at room | 60 minutes at 22° C. |
| | temperature | |
| Lysosome | 10 minutes at room | 120 minutes at 22° C. |
| | temperature | |
| Recycling Endosome | 15 minutes on ice | 15 minutes at room |
| | | temperature |

Experiments with S2R+ Cells:

Before pulse, 2 μM of Clensor^{Tf} is incubated in 1×M1 of pH of 7.2 containing 1.5 mg/mL BSA and 2mg/mL D-glucose for 30 minutes at Room Temperature.

Chloride Measurement using Clensor^{Tf}:

1. S2R+ cells are washed with 1×M1 buffer supplemented with BSA (1.5 mg/mL) and D-glucose (2 mg/mL) for couple of times.

2. The cells are incubated with 2 μM Clensor^{Tf}_{A647} for 15 minutes on ice.

3. Labeling mixture is removed and washed 3 to 4 times with 1×M1 buffer supplemented with BSA (1.5 mg/mL) and D-glucose (2 mg/mL).

4. Cells are chased for 15 minutes at room temperature and quickly transferred to ice.

5. Surface bound probes are stripped using stripping buffer at pH of 4.5* for 10 minutes in ice.

6. Cells are further washed 3 times with M1.

7. Cells are fixed for 10 minutes using 2.5% PFA in 1×M1.

8. Cells are imaged in M1 medium.

Competition Experiment with Excess Unlabeled Transferrin:

1. S2R+ cells are washed with 1×M1 buffer supplemented with BSA (1.5 mg/mL) and D-glucose (2 mg/mL) for couple of times.

2. Cells are incubated with

a) 1 μM Clensor-BAC

b) 1μM Clensor^{Tf}-BAC

c) 1 μM Clensor^{Tf}-BAC+25 μM unlabeled Transferrin or 100 μg/50 μl unlabeled Transferrin for 15 minutes on ice.

3. Labeling mixture is removed and washed 3 to 4 times with 1×M1 buffer supplemented with BSA (1.5 mg/mL) and D-glucose (2 mg/mL).

4. Cells are chased for 15 minutes at room temperature and quickly transferred to ice.

5. Surface bound probes are stripped using stripping buffer at pH of 4.5* for 10 minutes in ice.

6. Cells are further washed 3 times with 1×M1.

7. Cells are fixed for 10 minutes using 2.5% PFA in M1.

8. Cells are imaged in M1 medium.

Colocalization Experiment with Labeled Transferrin:

1. S2R+ cells are washed with 1×M1 buffer supplemented with BSA (1.5 mg/mL) and D-glucose (2 mg/mL) for couple of times.

2. Cells are incubated with 1 μM Clensor^{Tf}-BAC+100 nM Transferrin-A₅₆₈ for 15 minutes on ice.

3. Labeling mixture is removed and washed 3 to 4 times with 1×M1 buffer supplemented with BSA (1.5 mg/mL) and D-glucose (2 mg/mL).

4. Cells are chased for 15 minutes at room temperature and quickly transferred to ice.

5. Surface bound probes are stripped using stripping buffer at pH of 4.5* for 10 minutes in ice.

6. Cells are further washed 3 times with M1.

7. Cells are fixed for 10 minutes using 2.5% PFA in 1×M1.

8. Cells are imaged in M1 medium.

Chloride Clamping Experiment in S2R+ Cells:

1. S2R+ cells are washed with 1×M1 buffer supplemented with BSA (1.5 mg/mL) and D-glucose (2 mg/mL) for couple of times.

2. Cells are incubated with 2 μM Clensor^{Tf}_{A647} for 15 minutes on ice.

3. Labeling mixture is removed and washed 3 to 4 times with 1×M1 buffer supplemented with BSA (1.5 mg/mL) and D-glucose (2 mg/mL).

4. Cells are chased for 15 minutes at room temperature and quickly transferred to ice.

5. Surface bound probes are stripped using stripping buffer at pH of 4.5* for 10 minutes in ice.

6. Cells are further washed 3 times with 1×M1.

7. Cells are fixed for 20 minutes using 2.5% PFA in 1×M1.

8. Cells are further washed 3 times with 1×M1.

9. Cells are incubated in Chloride ion (Cl⁻) clamping solution (120 mM KCl/KNO₃, 20 mM NaCl/NaNO₃, 1 mM CaCl₂/Ca(NO₃)₂, 1 mM MgCl₂/Mg(NO₃)₂, and 10 mM HEPES of pH 7.2, with Chloride ion (Cl⁻) concentration from 0-80 mM by replacing Cl⁻ with NO₃⁻) of different Chloride ion (Cl⁻) concentration containing Nigericin (10 μM)+Valinomycin (10 μM)+CCCP (5 μM)+Monensin (10 μM)+Bafilomycin (200 nM) for 3 hours at Room Temperature.

10. Cells are imaged in the same buffer.

*Stripping buffer composition: 160 mM Sodium ascorbate+40 mM Ascorbic acid+1 mM MgCl₂+1 mM CaCl₂, pH of 4.5

1×M1 buffer: 150 mM NaCl, 5 mM KCl, 1 mM CaCl₂, 1 mM MgCl₂ and 20 mM HEPES of pH of 7.2.

Further, any aptamer that recognizes a cell surface protein is used to target the sensor of the present disclosure, for e. g. anti-EGFR, anti-HER3, anti-RET etc.

FIG. 11b provides a representative image of S2R+ cells pulsed with Clensor^{Tf} and FIG. 11c depicts results of a competition experiment with excess unlabeled transferrin. It is derived from the results of FIG. 11c) that Clensor^{Tf} transports via Transferrin pathway, as unlabelled transferrin competes out Clensor^{Tf}. Table 8 of the present disclosure provides the intensity of the internalized sensor as measured by Autofluorescence, Clensor, Clensor^{Tf} and Clensor^{Tf}+ unlabeled transferrin.

TABLE 8

| | Whole cell Fluorescence intensity in | |
|---|---|---|
| Mode | Alexa647 channel (A.U.) | |
| Autofluorescence (AF) | 62601.15385 ± | 6444.11539 |
| Clenso^{Tf} | 1.88935E6 ± | 389915.18563 |
| Clensor^{Tf} + unlabeled transferrin | 185952.64286 ± | 37092.27892 |
| Clensor | 47851.81818 ± | 6253.17033 |

### Example 8—Measurement of Chloride Concentration in Hemocytes and S2R+ Cells

In an embodiment of the present disclosure, Clensor and Clensor^{Tf} is targeted to various compartments inside S2R+ cells and Hemocytes. In an embodiment of the present disclosure, Clensor and Clensor^{Tf} report very small change in chloride concentration under both chemical and genetic perturbation of chloride transport. Table 9 summarizes chloride concentrations (mean±SEM) measured in different compartments of S2R+ cells and Hemocytes.

TABLE 9

| | [Cl⁻]_{EE} (mM) | [Cl⁻]_{LE} (mM) | [Cl⁻]_{LY} (mM) | [Cl⁻]_{RE} (mM) |
|---|---|---|---|---|
| CS hemocytes | 37.1 ± 1.6 | 60.4 ± 2 | 108.5 ± 1.4 | |
| DmClC-b | 37.7 ± 2 | 52.0 ± 1.9 | 70.9 ± 2.8 | |
| RNAi | | | | |
| hemocytes | | | | |
| DmClC-b | 37.9 ± 1.4 | 51.9 ± 3.9 | 58.6 ± 4.1 | |
| Mutant | | | | |
| hemocytes | | | | |
| DmClC-c | 28.7 ± 0.13 | 60.4 ± 2.7 | 113.9 ± 5.3 | |
| RNAi | | | | |
| hemocytes | | | | |
| DmClC-c | | | | |
| Mutant | | | | |
| hemocytes | | | | |
| S2R+ cells | | | | 39.9 ± 1.2 |
| S2R+ cells | | | | 33.1 ± 1.5 |
| DmClC-c | | | | |
| RNAi | | | | |
| S2R+ cells | | | | 39.1 ± 0.7 |
| DmClC-b | | | | |
| RNAi | | | | |

In the table provided above:
CS Hemocytes—hemocytes of wild type strain of Drosophila.
DmClC RNAi hemocytes—hemocytes with RNAi knockdown of DMClC-b and DMClC-c gene respectively.
DmClC mutant hemocytes—hemocytes with DmClC-b and DmClC-c gene disrupted by mutagenesis.
S2R+ cells—wild type S2R+ cells of Drosophila.
S2R+ cells DmClC−RNAi—S2R+ cells with RNAi knockdown of DMClC-b and DMClC-c gene respectively.

It is derived from the above presented table that if DmClC-c is absent, there is lesser chloride accumulation in early endosomes and recycling endosomes. Further, if DmClC-b is absent, there is lesser chloride accumulation in late endosomes and lysosomes.

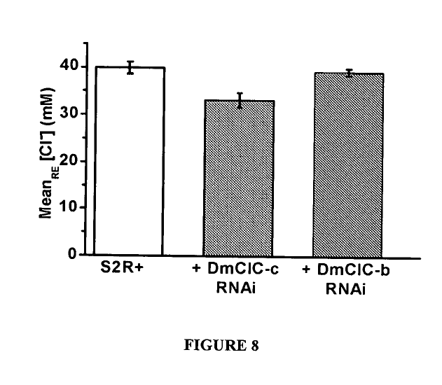

FIG. 8 of the present disclosure depicts measurement of Chloride ion concentration in recycling endosomes of S2R⁺ cells and in the background of RNAi knock down of DmClC-c and DmClC-b.

Table 10 of the present disclosure shows the chloride concentration measured in endosomal compartments of Drosophila hemocytes, under chemical perturbation, using Clensor. Here, the hemocytes are treated with a non-specific chloride ion channel blocker NPPB (5-nitro-2-(3-phenylpropylamino)-benzoate).

TABLE 10

| | Mean [Cl⁻] ± s.e.m (mM) | |
|---|---|---|
| Endosomes (min) | −NPPB | +NPPB |
| EE (5) | 37.1 ± 1.6 | 9.3 ± 1.5 |
| LE (60) | 60.4 ± 2 | 33.8 ± 2.5 |
| LY (120) | 108.5 ± 1.4 | 86.5 ± 3.5 |

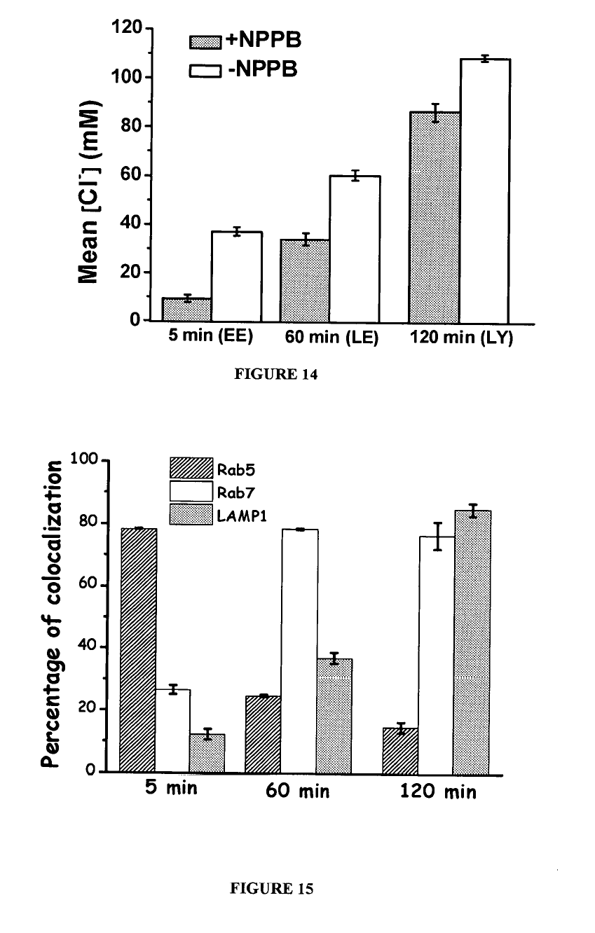

FIG. 14 of the present disclosure depicts measured chloride concentrations in the endosomal compartments with and without chloride channel blocker NPPB. It is derived from FIG. 14 that the Chloride ion concentration decreases in the presence of NPPB in each compartment.

In the table and figure presented above, it is observed that in different endosomal compartments, chloride accumulation goes down. It is derived from the results of this table and figure that Clensor specifically measures chloride concentration. This is concluded because, when Chloride ion concentration in a compartment is chemically perturbed, Clensor responds accordingly by providing the change in concentration. Thus, Clensor reports change in Chloride concentration due to impaired Chloride transport.

The present disclosure's design strategy is extended to develop sensor for any ion by using the relevant ion-sensitive dye.

### Example 9—Competition Experiment with mBSA

In an embodiment of the present disclosure, an experiment is performed to depict that Clensor is transported through the Anionic Ligand Binding Receptor (ALBR) in Drosophila hemocytes. For this protocol, mBSA—maleylated BSA which is negatively charged and is a known ligand for ALBR; is used. The internalization of Clensor is competed out by using excess of mBSA. It is observed that, in the presence of excess of mBSA, Clensor does not bind to the ALBR receptor and does not get internalised. Thus, it is determined that, Clensor is competed out by excess of mBSA, and thus it is transported by the ALBR receptor.

Protocol: Three different types of dishes containing Drosophila hemocytes are prepared. The first dish is incubated with a mixture of about 1 μM Clensor_{A647} and 10 fold excess of maleylated BSA (+mBSA) for about 5 minutes. The second dish is incubated with Clensor_{A647} alone (−mBSA) for about 5 minutes, and the third dish containing unlabeled cells is imaged to measure the contribution of auto-fluorescence (AF). The cells are then chased for 5 minutes to allow receptor internalization, washed 3 times with 1×L1 and then subjected to imaging under a wide-field microscope. The whole cell intensity in Alexa 647 channel is quantified for about 50 cells per dish. The mean intensity of all the three dishes, normalized with respect to the mean intensity in the cells pulsed with Clensor_{A647} alone (-mBSA) and is presented as the fraction of Clensor internalized.

Results and conclusion: Nucleic acid based sensors get endocytosed in Drosophila hemocytes through the ALBR (Anionic Ligand Binding Receptor) pathway. Nucleic acid based sensors act as anionic ligands due to the presence of the negatively charged phosphate backbone of dsDNA. Therefore, Drosophila hemocytes are chosen as a model system to measure the intracellular performance of Clensor.

To track the ALBR pathway in these cells, Clensor, which is molecularly programmed with a large dsDNA domain targeting to the ALBR pathway is used. It is known that Drosophila hemocytes internalize maleylated BSA (mBSA), a polyanionic ligand via the ALBR pathway. To confirm that Clensor is indeed internalized via this pathway, a competition experiment is performed with excess mBSA. The total fluorescence intensity of cells in Alexa 647 channel is quantified, normalized with respect to the fluorescence in untreated cells and presented as the fraction of Clensor_{A647} internalized.

FIG. 10b of the present disclosure provides a graph depicting the fraction of Clensor internalised in the presence and absence of mBSA. It shows that Clensor is efficiently competed out in the presence of excess mBSA, indicating that it is internalized via the ALBR pathway.

### Example 10—Experiment with Bafilomycin

The fluorescence of Chloride sensitive fluorophore—BAC decreases with increase of Chloride ion concentration. This experiment is performed to determine that the decrease in fluorescence of fluorophore BAC of Clensor, is due to increase of Chloride ion concentration, and not due to factors such as photo-bleaching of the fluorophore. This experiment provides that when the Chloride ion concentration is reduced in the cellular compartments, the fluorescence intensity of BAC is recovered.

The integrity of Clensor and the photostability of BAC is confirmed in the intracellular environment over 120 minutes of endosomal maturation, using the following assay.

Bafilomycin is used to perturb endosomal Chloride ion concentration [Cl⁻] and these perturbations are followed by changes in R/G ratios in Clensor labeled endosomes of Drosophila hemocytes. pH is simultaneously measured in this system by pulsing with about 2 mg/mL 10 kDa FITC-dextran (FD10) for about 5 minutes at room temperature and chased for about 120 minutes at about 20° C. The cells are then incubated with 1×M1 buffer containing about 200 nM Bafilomycin followed by a 120 minute chase at about 20° C. in the same buffer.

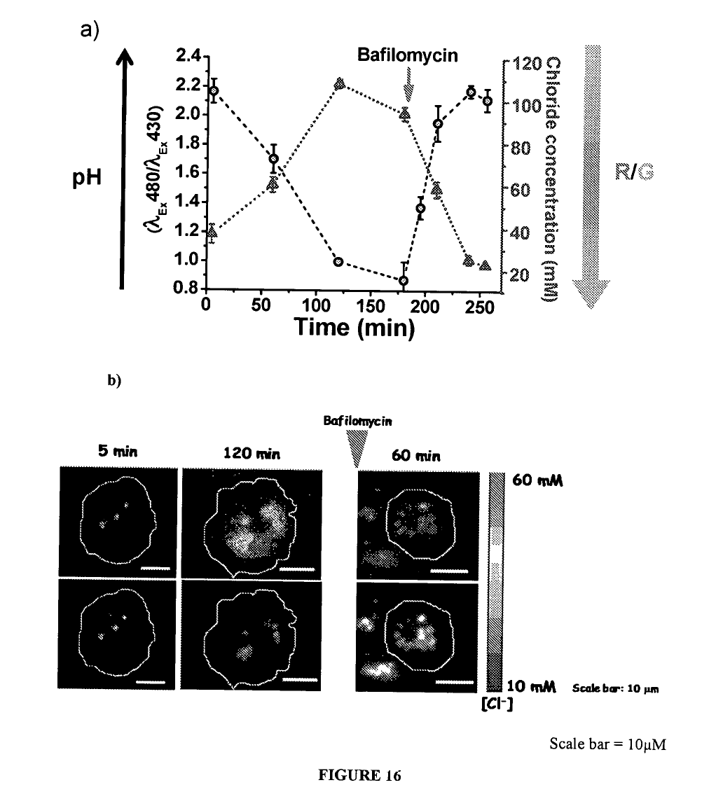

Intracellular pH is measured by the Dual Excitation Method. λ_{Ex}480/λ_{Ex} 430 emission ratios show about 2.1 fold decrease in pH as a function of time till about 120 minutes post endocytosis, indicating the expected decrease in pH in hemocytes (FIG. 16a, black). Upon Bafilomycin treatment, endosomal pH is reversed as given by gradual increase (˜2.2 fold) in λ_{Ex}480/λ_{Ex} 430 emission ratios (FIG. 16a, black). Independently, changes in Chloride ion concentration [Cl⁻] are mapped by pulsing hemocytes with Clensor for about 5 minutes at room temperature and followed by a 120 min chase at about 20° C. It is observed that there is a decrease in R/G ratios as a function of time till 120 min post endocytosis, corresponding to a change in mean endosomal Chloride ion concentration [Cl⁻] from about 37.1 mM to about 108.5 mM that then remain quite constant till about 180 minutes, when Bafilomycin is added in the external buffer (FIG. 16a, blue).

After Bafilomycin treatment, R/G ratios gradually decrease, indicating the reversal of endosomal Chloride ion concentration [Cl⁻] (FIG. 16a, blue). Here the decrease in R/G ratios or the recovery of BAC fluorescence indicates that reduction in BAC fluorescence is not due to photo-bleaching but due to collisional quenching by Chloride ion (Cl⁻) present in the lumen of endosomal compartment. Additionally, co-localization of BAC and Alexa 647 in the endosomal compartments during the time course of this experiment indicates the integrity of Clensor. To overcome the issue of photobleaching, same dishes are not imaged continuously. Instead, multiple dishes are chased for different time periods with or without Bafilomycin treatment over the full duration of the assay.

Thus, in this experiment, when the cellular compartments of Drosophila hemocytes are treated with Bafilomycin, it reverses the Chloride ion concentration and pH in the compartment. The Bafilomycin treated cells show that the pH reverts to its initial values and fluorescence of BAC is increasing. This experiment shows that the fluorophore of Clensor reports environmental Chloride ion concentration and this causes the decrease in the Fluorescence of BAC.

FIG. 16 depicts the integrity of Clensor during endosomal maturation. FIG. 16a of the present disclosure depicts change in chloride concentrations and pH of the endosomal compartments during endosomal maturation with time. The red arrow indicates the point of Bafilomycin addition. Chloride concentrations and pH values are monitored after Bafilomycin treatment. The hemocytes are pulsed with Clensor (about 2 μM, 50 μL) and 10 kDa FITC-dextran (about 2 mg/mL, 50 μL) in two independent experiments for 5 minutes and imaged at indicated time points. Endosomal pH is monitored by ratios of the intensity at 530 nm when the FITC is excited at 480 nm and 430 nm (λₑₓ480/λₑₓ 430).

After the initial chase period of 120 minutes, V-ATPase inhibitor 200 nM Bafilomycin is added externally, shown by red arrow head. The plot shows endosomal Chloride ion concentration [Cl⁻] (blue trace) and (λₑₓ480/λₑₓ430) ratios indicate endosomal pH as a function of time (black trace).

FIG. 16b depicts the representative images of hemocytes pulsed with Clensor before and after Bafilomycin treatment. The representative pseudocolour R/G map of hemocytes pulsed with Clensor (about 2 mM, 50 mL) and imaged at the indicated chase times before and after addition of Bafilomycin (about 200 nM) is shown by red arrow head Thus, FIG. 16 indicates that the functional and structural integrity of Clensor is preserved post internalization inside cells over duration of endosomal maturation.

### Example 11—Chloride Ion Concentration in Lifetime Measurement Mode

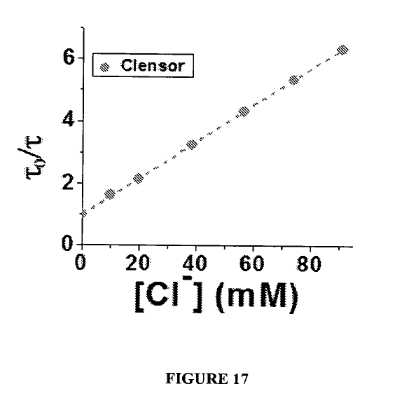

In an embodiment of the present disclosure, in vitro lifetime measurement of Clensor as a function of different chloride concentration is studied. In FIG. 17 of the present disclosure, a graph representing the Chloride ion concentration v/s the ratio of τ₀/τ. In the graph, τ₀ is the lifetime of BAC in absence of added chloride and τ is the lifetime of BAC in presence of different amount of added chloride in the solution. This figure depicts that Clensor shows measureable lifetime change in the physiological range of chloride concentration. Hence it is used to quantify chloride in lifetime mode as well.

Table 11 summarizes the change in lifetime of Clensor (in nanoseconds) as a function of chloride concentration.

TABLE 11

| [Cl⁻] (mM) | Clensor |
|---|---|
| 0 | 7.40 ± 0.01 |
| 9.9 | 4.54 ± 0.03 |
| 19.6 | 3.45 ± 0.07 |
| 38.4 | 2.28 ± 0.02 |
| 56.6 | 1.72 ± 0.01 |
| 74.1 | 1.39 ± 0.01 |
| 90.9 | 1.17 ± 0.00 |

Applications of the Nucleic Acid Based Sensor of the Present Disclosure:

1. Clensor is quantitative and targetable. So, it is used to measure chloride ion concentration in different intracellular compartments to elucidate the role of chloride along a specific pathway. Clensor is targeted to two different pathways: endosomal-lysosomal pathway and recycling pathway. Chloride ion measurement has also been done in these pathways.

2. Primary advantage of Clensor is that it allows assaying of chloride ion channel functioning in chemical space (chloride concentration) in the natural environment rather than conventional electrical space (conductivity) on isolated membranes or plasma membrane. Therefore it is used to study the localization and function of chloride ion transporters in cellulis. It is used as a sensor for assays that report on structure-function relationships in chloride transporters. Using Clensor, the present disclosure pin points the localization and function of a Drosophila CLC family chloride channels/transporter.

3. Altered chloride concentration leads to several diseases. Clensor is used to pin point the intracellular location of altered chloride concentration. Thus, it is used for screens that detect the cause of the disease or to diagnose it.

4. Additionally, there are genetic diseases caused by mutations in chloride channels (channelopathies)—the most common being Cystic Fibrosis, which has a prevalence of about 1 in 2,000 Caucasians. Thus, Clensor has direct applications in screening for the drug molecules.

1-15. (canceled)

16. A nucleic acid based sensor comprising:
a) sensing module comprising Peptide Nucleic Acid (PNA) strand and target sensitive molecule;
b) normalizing module comprising nucleic acid sequence complementary to the PNA strand and target insensitive fluorophore; and
c) targeting module comprising nucleic acid sequence complementary to the nucleic acid sequence of the normalizing module, optionally with aptamer.

17. A method of obtaining the nucleic acid based sensor as claimed in claim 16, said method comprising acts of:
a) obtaining sensing module by conjugating target sensitive molecule to Peptide Nucleic Acid (PNA) strand;
b) obtaining normalizing module by conjugating target insensitive fluorophore to nucleic acid sequence complementary to the PNA strand of the sensing module;
c) obtaining targeting module comprising nucleic acid sequence complementary to the nucleic acid sequence of the normalizing module, and optionally conjugating with aptamer; and
d) combining the sensing, the normalizing and the targeting module to obtain the nucleic acid based sensor.

18. A method of identifying and optionally quantifying target in a sample, said method comprising acts of:
a) contacting and incubating the sample with nucleic acid based sensor as claimed in claim 16;
b) identifying the target by determining change in fluorescence level; and
c) optionally quantifying the target by determining the fluorescence ratio of target insensitive fluorophore to target sensitive molecule.

19. The sensor as claimed in claim 16, wherein the targeting module comprises nucleic acid sequence selected from the group comprising DNA, RNA and PNA or any combinations thereof, preferably a combination of DNA and RNA; wherein the normalizing module comprises nucleic acid sequence selected from the group comprising DNA, RNA and PNA or any combinations thereof, preferably DNA; and wherein the nucleic acid based sensor is for detecting target selected from the group comprising Cl⁻, Ca²⁺, Mg²⁺, Zn²⁺, Cu²⁺, Fe²⁺, Pb²⁺, Cd²⁺, Hg²⁺, Ni²⁺, Co²⁺, H⁺, Na⁺, K⁺, F⁻, Br⁻, I⁻, Cyanide (CN⁻), Nitrate (NO³⁻), Nitrite (NO²⁻), Nitric oxide, Phosphate (PO³⁻), Pyrophosphate (P₂O₇⁴⁻) and Reactive Oxygen species, preferably chloride (Cl⁻) ion.

20. The sensor as claimed in claim 16, wherein the target sensitive molecule is selected from the group comprising SPQ (6-methoxy-N-(3-ulphopropyl) quinolinium), MACA (10-methylacridinium-9-carboxamide), MADC (10-methylacridinium-9-N,N-dimethylcarboxamide), MAMC (N-methylacridinium-9-methylcarboxylate), DMAC (2, 10-Dimethylacridinium-9-carboxaldehyde), MAA (N-methyl-9-aminoacridinium), 6-methoxy-N-(4-sulphobutyl) quinolinium, N-dodecyl-6-methoxy-quinolinium iodide, 6-methyl-N-(3-sulphopropyl) quinolinium, 6-methoxy-N-(8-octanoic acid) quinolinium bromide, 6-methoxy-N-(8-octanoic acid) quinoliniumtetraphenyl borate, 6-methyl-N-(methyl) quinolinium bromide, 6-methyl-N-(methyl) quinolinium iodide, N, N′-dimethyl-9-9′-bisacridinium and 10,10′-Bis[3-carboxypropyl]-9,9′-biacridiniumDinitrate (BAC) or modifications and derivatives thereof, preferably 10,10′-Bis[3-carboxypropyl]-9,9′-biacridiniumDinitrate (BAC); the target insensitive fluorophore is selected from the group comprising Alexafluor 568, Alexafluor 594 and Alexa 647, preferably Alexa 647; and ratio of the target-sensitive molecule and the target insensitive fluorophore is 1:1.

21. The sensor as claimed in claim 16, wherein the Peptide Nucleic Acid (PNA) strand comprises Seq ID No.1; the normalizing module comprises Seq ID No.2; and the targeting module comprises sequence selected from the group comprising Seq ID No.3 and Seq ID No.4; and wherein the aptamer targets the sensor to specific location in cell and is selected from the group comprising DNA, RNA and PNA or any combinations thereof.

22. The sensor as claimed in claim 16, wherein the aptamer is RNA aptamer that specifically binds to Human Transferrin Receptor; and wherein the RNA aptamer comprises Seq ID No. 13.

23. A method of targeting nucleic acid based sensor as claimed in claim 16, said method comprising acts of:
a) obtaining nucleic acid based sensor by method as claimed in claim 17;
b) adding the sensor to cell for cellular uptake, to obtain a cell with the sensor;
c) incubating the cell obtained in step b) for the nucleic acid based sensor to follow targeted cellular pathway within the cell.

24. The method as claimed in claim 23, wherein the cell is selected from the group comprising prokaryotic cell and eukaryotic cell; and wherein the targeting module of the nucleic acid based sensor is engineered to target the nucleic acid based sensor to follow cellular pathway within the cell.

25. A kit for obtaining or targeting the nucleic acid based sensor as claimed in claim 16 or identifying and optionally quantifying target in a sample, said kit comprising components selected from the group comprising sensing module, targeting module, normalizing module, nucleic acid based sensor, cell, sample and instructions manual or any combinations thereof.

26. A method of assembling the kit as claimed in claim 25 , said method comprising act of combining components selected from the group comprising sensing module, targeting module, normalizing module, nucleic acid based sensor, cell, sample and instructions manual or any combinations thereof.

27. The method as claimed in claim 17, wherein the targeting module comprises nucleic acid sequence selected from the group comprising DNA, RNA and PNA or any combinations thereof, preferably a combination of DNA and RNA; wherein the normalizing module comprises nucleic acid sequence selected from the group comprising DNA, RNA and PNA or any combinations thereof, preferably DNA; and wherein the nucleic acid based sensor is for detecting target selected from the group comprising Cl⁻, Ca²⁺, Mg²⁺, Zn²⁺, Cu²⁺, Fe²⁺, Pb²⁺, Cd²⁺, Hg²⁺, Ni²⁺, Co²⁺, H⁺, Na⁺, K⁺, Br⁻, I⁻, Cyanide (CN⁻), Nitrate (NO³⁻), Nitrite (NO²⁻), Nitric oxide, Phosphate (PO³⁻), Pyrophosphate (P₂O₇⁴⁻) and Reactive Oxygen species, preferably chloride (Cl⁻) ion.

28. The method as claimed in claim 17, wherein the target sensitive molecule is selected from the group comprising SPQ (6-methoxy-N-(3-ulphopropyl) quinolinium), MACA (10-methylacridinium-9-carboxamide), MADC (10-methylacridinium-9-N,N-dimethylcarboxamide), MAMC (N-methylacridinium-9-methylcarboxylate), DMAC (2, 10-Dimethylacridinium-9-carboxaldehyde), MAA (N-methyl-9-aminoacridinium), 6-methoxy-N-(4-sulphobutyl) quinolinium, N-dodecyl-6-methoxy-quinolinium iodide, 6-methyl-N-(3-sulphopropyl) quinolinium, 6-methoxy-N-(8-octanoic acid) quinolinium bromide, 6-methoxy-N-(8-octanoic acid) quinoliniumtetraphenyl borate, 6-methyl-N-(methyl) quinolinium bromide, 6-methyl-N-(methyl) quinolinium iodide, N, N′-dimethyl-9-9′-bisacridinium and 10,10′-Bis[3-carboxypropyl]-9,9′-biacridiniumDinitrate (BAC) or modifications and derivatives thereof, preferably 10,10′-Bis[3-carboxypropyl]-9,9′-biacridiniumDinitrate (BAC); the target insensitive fluorophore is selected from the group comprising Alexafluor 568, Alexafluor 594 and Alexa 647, preferably Alexa 647; and ratio of the target-sensitive molecule and the target insensitive fluorophore is 1:1.

29. The method as claimed in claim 17, wherein the Peptide Nucleic Acid (PNA) strand comprises Seq ID No.1, the normalizing module comprises Seq ID No.2, and the targeting module comprises sequence selected from the group comprising Seq ID No.3 and Seq ID No.4; and wherein the aptamer targets the sensor to specific location in cell and is selected from the group comprising DNA, RNA and PNA or any combinations thereof.

30. The method as claimed in claim 17, wherein the aptamer is RNA aptamer that specifically binds to Human Transferrin Receptor; and wherein the RNA aptamer comprises Seq ID No. 13.

31. The method as claimed in claim 18, wherein the nucleic acid based sensor is for detecting target selected from the group comprising Cl⁻, Ca²⁺, Mg²⁺, Zn²⁺, Cu²⁺, Fe²⁺, Pb²⁺, Cd²⁺, Hg²⁺, Ni²⁺, Co²⁺, H⁺, Na⁺, K⁺, F⁻, Br⁻, I⁻, Cyanide (CN⁻), Nitrate (NO³⁻), Nitrite (NO²⁻), Nitric oxide, Phosphate (PO³⁻), Pyrophosphate (P₂O₇⁴⁻) and Reactive Oxygen species, preferably chloride (Cl⁻) ion.

32. The method as claimed in claim 18, wherein the target sensitive molecule is selected from the group comprising SPQ (6-methoxy-N-(3-ulphopropyl) quinolinium), MACA (10-methylacridinium-9-carboxamide), MADC (10-methylacridinium-9-N,N-dimethylcarboxamide), MAMC (N-methylacridinium-9-methylcarboxylate), DMAC (2, 10-Dimethylacridinium-9-carboxaldehyde), MAA (N-methyl-9-aminoacridinium), 6-methoxy-N-(4-sulphobutyl) quinolinium, N-dodecyl-6-methoxy-quinolinium iodide, 6-methyl-N-(3-sulphopropyl) quinolinium, 6-methoxy-N-(8-octanoic acid) quinolinium bromide, 6-methoxy-N-(8-octanoic acid) quinoliniumtetraphenyl borate, 6-methyl-N-(methyl) quinolinium bromide, 6-methyl-N-(methyl) quinolinium iodide, ; N, N′-dimethyl-9-9′-bisacridinium and 10,10′-Bis[3-c arboxypropyl]-9,9′-biacridiniumDinitrate (BAC) or modifications and derivatives thereof, preferably 10,10′-Bis[3-carboxypropyl]-9,9′-biacridiniumDinitrate (BAC); the target insensitive fluorophore is selected from the group comprising Alexafluor 568, Alexafluor 594 and Alexa 647, preferably Alexa 647; and ratio of the target-sensitive molecule and the target insensitive fluorophore is 1:1.

33. The method as claimed in claim 18, wherein the sample is biological sample selected from the group comprising cell, cell extract, cell lysate, tissue, tissue extract, bodily fluid, serum, blood and blood product.
